(19) United States
(12) Patent Application Publication
(10) Pub. No.: US 2010/0304464 A1
(43) Pub. Date: Dec. 2, 2010

(54) STABLE, FUNCTIONAL CHIMERIC CELLOBIOHYDROLASES

(75) Inventors: Frances H. Arnold, La Canada, CA (US); Pete Heinzelman, Pasadena, CA (US); Jeremy Minshull, Menlo Park, CA (US); Sridhar Govindarajan, Redwood City, CA (US); Alan Villalobos, San Francisco, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: CALIFORNIA INSTITUTE OF TECHNOLOGY, Pasadena, CA (US)

(21) Appl. No.: 12/723,597

(22) Filed: Mar. 12, 2010

Related U.S. Application Data
(60) Provisional application No. 61/167,003, filed on Apr. 6, 2009.

(51) Int. Cl. C12N 9/42; C07H 21/00; C12N 15/63; C12N 1/00

(52) U.S. Cl. 435/209; 536/23.2; 435/320.1; 435/243

(57) ABSTRACT

The present disclosure relates to CBH II chimera fusion polypeptides, nucleic acids encoding the polypeptides, and host cells for producing the polypeptides.

### CROSS REFERENCE TO RELATED APPLICATIONS

The application claims priority under 35 U.S.C. §119 to U.S. Provisional Application Ser. Nos. 61/205,284, filed Jan. 16, 2009, and 61/167,003, filed, Apr. 6, 2009, the disclosure of which is incorporated herein by reference.

### STATEMENT REGARDING FEDERALLY SPONSORED RESEARCH

The U.S. Government has certain rights in this invention pursuant to Grant No. GM068664 awarded by the National Institutes of Health and Grant No. DAAD19-03-0D-0004 awarded by ARO—US Army Robert Morris Acquisition Center.

### TECHNICAL FIELD

The present disclosure relates to biomolecular engineering and design, and engineered proteins and nucleic acids.

### BACKGROUND

The performance of cellulase mixtures in biomass conversion processes depends on many enzyme properties including stability, product inhibition, synergy among different cellulase components, productive binding versus nonproductive adsorption and pH dependence, in addition to the cellulose substrate physical state and composition. Given the multivariate nature of cellulose hydrolysis, it is desirable to have diverse cellulases to choose from in order to optimize enzyme formulations for different applications and feedstocks.

### SUMMARY

The disclosure provides a chimeric polypeptide comprising at least two domains from two different parental cellobiohydrolase II (CBH II) polypeptides, wherein the domains comprise from N- to C-terminus: (segment 1)-(segment 2)-(segment 3)-(segment 4)-(segment 5)-(segment 6)-(segment 7)-(segment 8); wherein: segment 1 comprises a sequence that is at least 50-100% identical to amino acid residue from about 1 to about x₁ of SEQ ID NO:2 (“1”), SEQ ID NO:4 (“2”) or SEQ ID NO:6 (“3”); segment 2 comprises a sequence that is at least 50-100% identical to amino acid residue x₁ to about x₂ of SEQ ID NO:2 (“1”), SEQ ID NO:4 (“2”) or SEQ ID NO:6 (“3”); segment 3 comprises a sequence that is at least 50-100% identical to amino acid residue x₂ to about x₃ of SEQ ID NO:2 (“1”), SEQ ID NO:4 (“2”) or SEQ ID NO:6 (“3”); segment 4 comprises a sequence that is at least 50-100% identical to amino acid residue x₃ to about x₄ of SEQ ID NO:2 (“1”), SEQ ID NO:4 (“2”) or SEQ ID NO:6 (“3”); segment 5 comprises a sequence that is at least 50-100% identical to about amino acid residue x₄ to about x₅ of SEQ ID NO:2 (“1”), SEQ ID NO:4 (“2”) or SEQ ID NO:6 (“3”); segment 6 comprises a sequence that is at least 50-100% identical to amino acid residue x₅ to about x₆ of SEQ ID NO:2 (“1”), SEQ ID NO:4 (“2”) or SEQ ID NO:6 (“3”); segment 7 comprises a sequence that is at least 50-100% identical to amino acid residue x₆ to about x₇ of SEQ ID NO:2 (“1”), SEQ ID NO:4 (“2”) or SEQ ID NO:6 (“3”); and segment 8 comprises a sequence that is at least 50-100% identical to amino acid residue x₇ to about x₈ of SEQ ID NO:2 (“1”), SEQ ID NO:4 (“2”) or SEQ ID NO:6 (“3”); wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6;.x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6, wherein the chimeric polypeptide has cellobiohydrolase activity and improved thermostability and/or pH stability compared to a CBH II polypeptide comprising SEQ ID NO:2, 4, or 6. In one embodiment, segment 1 comprises amino acid residue from about 1 to about x₁ of SEQ ID NO:2 (“1”), SEQ ID NO:4 (“2”) or SEQ ID NO:6 (“3”) and having 1-10 conservative amino acid substitutions; segment 2 is from about amino acid residue x₁ to about x₂ of SEQ ID NO:2 (“1”), SEQ ID NO:4 (“2”) or SEQ ID NO:6 (“3”) and having about 1-10 conservative amino acid substitutions; segment 3 is from about amino acid residue x₂ to about x₃ of SEQ ID NO:2 (“1”), SEQ ID NO:4 (“2”) or SEQ ID NO:6 (“3”) and having about 1-10 conservative amino acid substitutions; segment 4 is from about amino acid residue x₃ to about x₄ of SEQ ID NO:2 (“1”), SEQ ID NO:4 (“2”) or SEQ ID NO:6 (“3”) and having about 1-10 conservative amino acid substitutions; segment 5 is from about amino acid residue x₄ to about x₅ of SEQ ID NO:2 (“1”), SEQ ID NO:4 (“2”) or SEQ ID NO:6 (“3”) and having about 1-10 conservative amino acid substitutions; segment 6 is from about amino acid residue x₅ to about x₆ of SEQ ID NO:2 (“1”), SEQ ID NO:4 (“2”) or SEQ ID NO:6 (“3”) and having about 1-10 conservative amino acid substitutions; segment 7 is from about amino acid residue x₆ to about x₇ of SEQ ID NO:2 (“1”), SEQ ID NO:4 (“2”) or SEQ ID NO:6 (“3”) and having about 1-10 conservative amino acid substitutions; and segment 8 is from about amino acid residue x₇ to about x₈ of SEQ ID NO:2 (“1”), SEQ ID NO:4 (“2”) or SEQ ID NO:6 (“3”) and having about 1-10 conservative amino acid substitutions. In yet another embodiment, the chimeric polypeptide has at least one segment selected from the following: segment 1 from SEQ ID NO:2; segment 6 from SEQ ID NO:6, segment 7 from SEQ ID NO:6 and segment 8 from SEQ ID NO:4. In yet another embodiment, the chimeric polypeptide can be described as having segments 1X₂X₃X₄X₅332, wherein X₂ comprises a sequence that is at least 50-100% identical to amino acid residue x₁ to about x₂ of SEQ ID NO:2 (“1”), SEQ ID NO:4 (“2”) or SEQ ID NO:6 (“3”); X₃ comprises a sequence that is at least 50-100% identical to amino acid residue x₂ to about x₃ of SEQ ID NO:2 (“1”), SEQ ID NO:4 (“2”) or SEQ ID NO:6 (“3”); X₄ comprises a sequence that is at least 50-100% identical to amino acid residue x₃ to about x₄ of SEQ ID NO:2 (“1”), SEQ ID NO:4 (“2”) or SEQ ID NO:6 (“3”); X₅ comprises a sequence that is at least 50-100% identical to about amino acid residue x₄ to about x₅ of SEQ ID NO:2 (“1”), SEQ ID NO:4 (“2”) or SEQ ID NO:6 (“3”). In yet a further embodiment, the chimeric polypeptide comprises a segment structure selected from the group consisting of 11113132, 21333331, 21311131, 22232132, 33133132, 33213332, 13333232, 12133333, 13231111, 11313121, 11332333, 12213111, 23311333, 13111313, 31311112, 23231222, 33123313, 22212231, 21223122, 21131311, 23233133, 31212111, 12222332 and 32333113. In one embodiment, the cimeric polypeptide comprises a segment structure selected from the group set forth in Table 1.

The disclosure also provides a polynucleotide encoding a polypeptide as described above. One of skill can readily determine the exact sequence desired using the degeneracy of the genetic code, by reference to the amino acid sequences herein and by reference to the polynucleotide sequences herein.

The disclosure also provides a vector comprising a polynucleotide of the disclosure as well as host cells comprising a polynucleotide or vector of the disclosure.

The disclosure provides an enzymatic preparation comprising a polypeptide described above.

The disclosure also provides a method of treating a biomass comprising cellulose, the method comprising contacting the biomass with a chimeric polypeptide as described above.

The disclosure provides a method of treating a biomass comprising cellulose, the method comprising contacting the biomass with a host cell comprising and expressing a polynucleotide and chimeric polypeptide of the disclosure, respectively.

The disclosure also provides a method of generating a thermostable chimeric cellobiohydrolase polypeptide, comprising recombining segments from at least 2 parental cellobiohydrolase polypeptide wherein the chimeric polypeptide comprises from N- to C-terminus 8 segments wherein: segment 1 comprises a sequence that is at least 50-100% identical to amino acid residue from about 1 to about x₁ of SEQ ID NO:2 (“1”), SEQ ID NO:4 (“2”) or SEQ ID NO:6 (“3”); segment 2 comprises a sequence that is at least 50-100% identical to amino acid residue x₁ to about x₂ of SEQ ID NO:2 (“1”), SEQ ID NO:4 (“2”) or SEQ ID NO:6 (“3”); segment 3 comprises a sequence that is at least 50-100% identical to amino acid residue x₂ to about x₃ of SEQ ID NO:2 (“1”), SEQ ID NO:4 (“2”) or SEQ ID NO:6 (“3”); segment 4 comprises a sequence that is at least 50-100% identical to amino acid residue x₃ to about x₄ of SEQ ID NO:2 (“1”), SEQ ID NO:4 (“2”) or SEQ ID NO:6 (“3”); segment 5 comprises a sequence that is at least 50-100% identical to about amino acid residue x₄ to about x₅ of SEQ ID NO:2 (“1”), SEQ ID NO:4 (“2”) or SEQ ID NO:6 (“3”); segment 6 comprises a sequence that is at least 50-100% identical to amino acid residue x₅ to about x₆ of SEQ ID NO:2 (“1”), SEQ ID NO:4 (“2”) or SEQ ID NO:6 (“3”); segment 7 comprises a sequence that is at least 50-100% identical to amino acid residue x₆ to about x₇ of SEQ ID NO:2 (“1”), SEQ ID NO:4 (“2”) or SEQ ID NO:6 (“3”); and segment 8 comprises a sequence that is at least 50-100. % identical to amino acid residue x₇ to about x₈ of SEQ ID NO:2 (“1”), SEQ ID NO:4 (“2”) or SEQ ID NO:6 (“3”); wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:2 or SEQ ID NO:3; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6; screening the chimeric polypeptide for the ability to hydrolyze cellulose at a temperature of about 63° C.

### BRIEF DESCRIPTION OF THE FIGURES

FIG. 1 shows SDS-PAGE gel of candidate CBH II parent gene yeast expression culture supernatants. Gel Lanes (Left-to-Right): 1-H. jecorina, 2-Empty vector, 3-H. insolens, 4-C. thermophilum, 5-H. jecorina (duplicate), 6-P. chrysosporium, 7-T. emersonii, 8-Empty vector (duplicate), 9-H. jecorina (triplicate). Numbers at bottom of gel represent concentration of reducing sugar (ug/mL) present in reaction after 2-hr, 50° C. PASC hydrolysis assay. Subsequent SDS-PAGE comparison with BSA standard allowed estimation of H. insolens expression level of 5-10 mg/L.

FIG. 2A-C shows illustrations of CBH II chimera library block boundaries. (A) H. insolens CBH II catalytic domain ribbon diagram with blocks distinguished by shading. CBH II enzyme is complexed with cellobio-derived isofagomine glycosidase inhibitor. (B) Linear representation of H. insolens catalytic domain showing secondary structure elements, disulfide bonds and block divisions denoted by black arrows. (C) Sidechain contact map denoting contacts (side chain heavy atoms within 4.5 Å) that can be broken upon recombination. The majority of broken contacts occur between consecutive blocks.

FIG. 3 shows a number of broken contacts (E) and number of mutations from closest parent (m) for 23 secreted/active and 25 not secreted/not active sample set chimeras.

FIG. 4 shows specific activity, normalized to pH 5.0, as a function of pH for parent CBH II enzymes and three thermostable chimeras. Data presented are averages for two replicates, where error bars for HJP1us and H. jeco denote values for two independent trials. 16-hr reaction, 300 ug enzyme/g PASC, 50° C., 12.5 mM sodium citrate/12.5 mM sodium phosphate buffer at pH as shown.

FIG. 5 shows long-time cellulose hydrolysis assay results (ug glucose reducing sugar equivalent/ug CBH II enzyme) for parents and thermostable chimeras across a range of temperatures. Error bars indicate standard errors for three replicates of HJPlus and H. insolens CBH II enzymes. 40-hr reaction, 100 ug enzyme/g PASC, 50 mM sodium acetate, pH 4.8.

FIG. 6 shows normalized residual activities for validation set chimeras after a 12-h incubation at 63° C. Residual activities for CBH II enzymes in concentrated culture supernatants determined in 2-hr assay with PASC as substrate, 50° C., 25 mM sodium acetate buffer; pH 4.8.

FIG. 7 Map for parent and chimera CBH II enzyme expression vector Yep352/PGK91-1-ss. Vector pictured contains wild type H. jecorina cel6a (CBH II enzyme) gene. For both chimeric and parent CBH II enzymes, the CBD/linker amino acid sequence following the ss Lys-Arg Kex2 site is: ASCSSVWGQCGGQNWSGPTCCASGSTCVYSND YYSQCLPGAASSSSSTRAASTTSRVSPTTSRSSSATPPPGSTTTRVPPVGSGTATYS (SEQ ID NO:8).

### DETAILED DESCRIPTION

As used herein and in the appended claims, the singular forms “a,” “and,” and “the” include plural referents unless the context clearly dictates otherwise. Thus, for example, reference to “a domain” includes a plurality of such domains and reference to “the protein” includes reference to one or more proteins, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of:”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

Recent studies have documented the superior performance of cellulases from thermophilic fungi relative to their mesophilic counterparts in laboratory scale biomass conversion processes, where enhanced stability leads to retention of activity over longer periods of time at both moderate and elevated temperatures. Fungal cellulases are attractive because they are highly active and can be expressed in fungal hosts such as Hypocrea jecorina (anamorph Trichoderma reesei) at levels up to 100 g/L in the supernatant. Unfortunately, the set of documented thermostable fungal cellulases is small. In the case of the processive cellobiohydrolase class II (CBH II) enzymes, fewer than 10 natural thermostable gene sequences are annotated in the CAZy database.

The majority of biomass conversion processes use mixtures of fungal cellulases (primarily CBH II, cellobiohydrolase class I (CBH I), endoglucanases and β-glucosidase) to achieve high levels of cellulose hydrolysis. Generating a diverse group of thermostable CBH II enzyme chimeras is the first step in building an inventory of stable, highly active cellulases from which enzyme mixtures can be formulated and optimized for specific applications and feedstocks.

SCHEMA has been used previously to create families of hundreds of active β-lactamase and cytochrome P450 enzyme chimeras. SCHEMA uses protein structure data to define boundaries of contiguous amino acid “blocks” which minimize <E>, the library average number of amino acid sidechain contacts that are broken when the blocks are swapped among different parents. It has been shown that the probability that β-lactamase chimera was folded and active was inversely related to the value of E for that sequence. The RASPP (Recombination as Shortest Path Problem) algorithm was used to identify the block boundaries that minimized <E>relative to the library average number of mutations, <m>. More than 20% of the ˜500 unique chimeras characterized from a β-lactamase collection comprised of 8 blocks from 3 parents (3⁸=6,561 possible sequences) were catalytically active. A similar approach produced a 3-parent, 8-block cytochrome P450 chimera family containing more than 2,300 novel, catalytically active enzymes. Chimeras from these two collections were characterized by high numbers of mutations, 66 and 72 amino acids on average from the closest parent, respectively. SCHEMA/RASPP thus enabled design of chimera families having significant sequence diversity and an appreciable fraction of functional members.

It has also been shown that the thermostabilities of SCHEMA chimeras can be predicted based on sequence-stability data from a small sample of the sequences. Linear regression modeling of thermal inactivation data for 184 cytochrome P450 chimeras showed that SCHEMA blocks made additive contributions to thermostability. More than 300 chimeras were predicted to be thermostable by this model, and all 44 that were tested were more stable than the most stable parent. It was estimated that as few as 35 thermostability measurements could be used to predict the most thermostable chimeras. Furthermore, the thermostable P450 chimeras displayed unique activity and specificity profiles, demonstrating that chimeragenesis can lead to additional useful enzyme properties. The disclosure demonstrates that SCHEMA recombination of CBH II enzymes can generate chimeric cellulases that are active on phosphoric acid swollen cellulose (PASC) at high temperatures, over extended periods of time, and broad ranges of pH.

Using the methods described herein a number of chimeric polypeptides having cellobiohydrolases activity were generated having improved characteristics compared to the wild-type parental CBH II proteins.

A diverse family of novel CBH II enzymes was constructed by swapping blocks of sequence from three fungal CBH II enzymes. Twenty-three of 48 chimeric sequences sampled from this set were secreted in active form by S. cerevisiae, and five have half-lives at 63° C. that were greater than the most stable parent. Given that this 48-member sample set represents less than 1% of the total possible 6,561 sequences, the disclosure provides hundreds of active chimeras, a number that extends well beyond the approximately twenty fungal CBH II enzymes in the CAZy database.

The approach of using the sample set sequence-stability data to identify blocks that contribute positively to chimera thermostability was validated by finding that all 10 catalytically active chimeras in the second CBH II validation set were more thermostable than the most stable parent, a naturally-thermostable CBH II from the thermophilic fungus, H. insolens. This disclosure demonstrates that a sample of 33 new CBH II enzymes that are expressed in catalytically active form in S. cerevisiae, 15 of which are more thermostable than the most stable parent from which they were constructed. These 15 thermostable enzymes are diverse in sequence, differing from each other and their closest natural homologs at as many as 94 and 58 amino acid positions, respectively.

Analysis of the thermostabilities of CBH II chimeras in the combined sample and validation sets indicates that the four thermostabilizing blocks identified; block 1 (i.e., domain 1), parent 1 (B1P1); block 6 (i.e., domain 6), parent 3 (B6P3); B7P3 and B8P2, make cumulative contributions to thermal stability when present in the same chimera. Four of the five sample set chimeras that are more thermostable than the H. insolens CBH II contain either two or three of these stabilizing blocks (Table 1). The ten active members of the validation set, all of which are more stable than the H. insolens enzyme, contain at least two stabilizing blocks, with five of the six most thermostable chimeras in this group containing either three or four stabilizing blocks.

Minimizing the number of broken contacts upon recombination (FIG. 2C) allows the blocks to be approximated as decoupled units that make independent contributions to the stability of the entire protein, thus leading to cumulative or even additive contributions to chimera thermostability. For this CBH II enzyme recombination, SCHEMA was effective in minimizing such broken contacts: whereas there are 303 total interblock contacts defined in the H. insolens parent CBH II crystal structure, the CBH II SCHEMA library design results in only 33 potential broken contacts. Given that the CBH II enzyme parents do not feature obvious structural subdomains, and only four of the eight blocks (1, 5, 7 and 8) resemble compact structural units, or modules, the low number of broken contacts demonstrates that the SCHEMA/RASPP algorithm is effective for cases in which the number of blocks appears greater than the number of structural subdivisions. As previously observed for β-lactamase and cytochrome P450 chimeras, low E values were predictive of chimera folding and activity. Although not used here, this relationship should be valuable for designing chimera sample sets that contain a high fraction of active members.

The disclosure also used chimera to determine if the pH stability could be improved in CBH II enzymes. Whereas the specific activity of H. jecorina CBH II declines sharply as pH increases above the optimum value of 5, HJP1us, created by substituting stabilizing blocks onto the most industrially relevant H. jecorina CBH II enzyme, retains significantly more activity at these higher pHs (FIG. 4). The thermostable 11113132 and 13311332 chimeras, and also the H. insolens and C. thermophilum CBH II cellulase parents, have even broader pH/activity profiles than HJP1us. The narrow pH/activity profile of H. jecorina CBH II has been attributed to the deprotonation of several carboxyl-carboxylate pairs, which destabilizes the protein above a pH of about 6. The substitution of parent 3 in block 7 (B7P3) in HJP1us changes aspartate 277 to histidine, eliminating the carboxyl-carboxylate pair between D277 and D316 (of block 8). Replacing D277 with the positively charged histidine may prevent destabilizing charge repulsion at nonacidic pH, allowing HJP1us to retain activity at higher pH than H. jecorina CBH II. The even broader pH/activity profiles of the remaining two thermostable chimeras and the H. insolens and C. thermophilum parent CBH II enzymes may be due to the absence of acidic residues at positions corresponding to the E57-E119 carboxyl-carboxylate pair of HJP1us and H. jecorina CBH II.

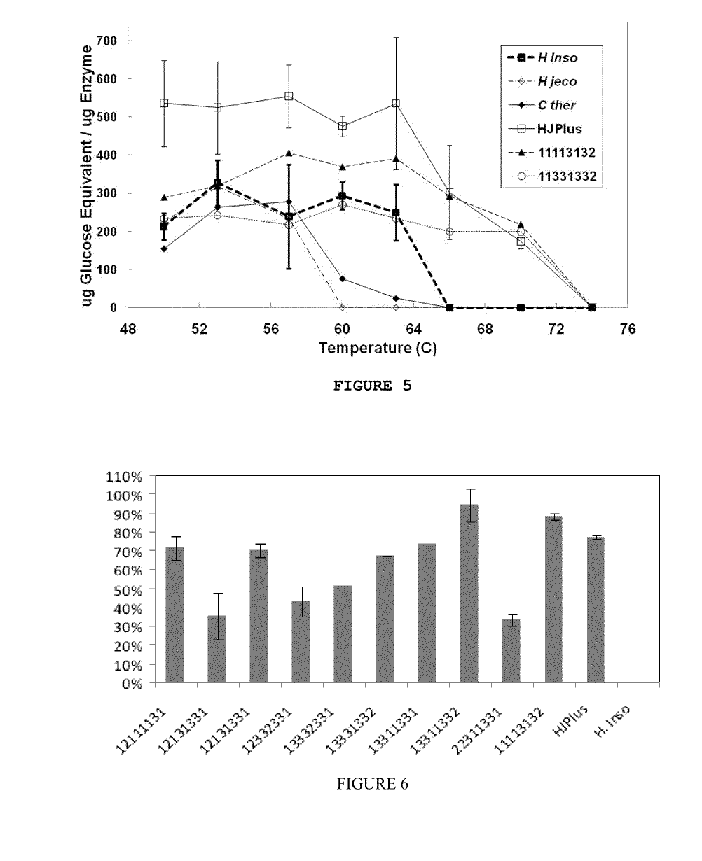

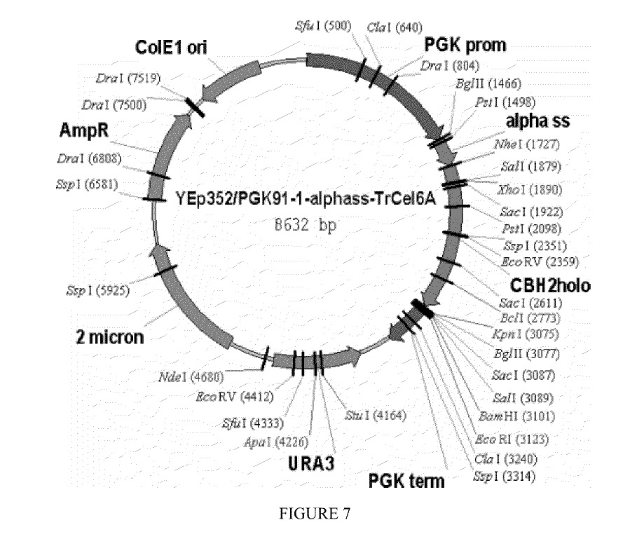

HJP1us exhibits both relatively high specific activity and high thermostability. FIG. 5 shows that these properties lead to good performance in long-time hydrolysis experiments: HJP1us hydrolyzed cellulose at temperatures 7-15° C. higher than the parent CBH II enzymes and also had a significantly increased long-time activity relative to all the parents at their temperature optima, bettering H. jecorina CBH II by a factor of 1.7. Given that the specific activity of the HJP1us chimera is less than that of the H. jecorina CBH II parent, this increased long-time activity can be attributed to the ability of the thermostable HJP1us to retain activity at optimal hydrolysis temperatures over longer reaction timer.

The other two thermostable chimeras shared HJP1us's broad temperature operating range. This observation supports a positive correlation between t_{1/2} at elevated temperature and maximum operating temperature, and suggests that many of the thermostable chimeras among the 6,561 CBH II chimera sequences will also be capable of degrading cellulose at elevated temperatures. While this ability to hydrolyze the amorphous PASC substrate at elevated temperatures bodes well for the potential utility of thermostable fungal CBH II chimeras, studies with more challenging crystalline substrates and substrates containing lignin will provide a more complete assessment of this novel CBH II enzyme family's relevance to biomass degradation applications.

“Amino acid” is a molecule having the structure wherein a central carbon atom is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the disclosure and may be referred to herein as “proteins.” In one embodiment of the disclosure, a stabilized protein comprises a chimera of two or more parental peptide segments.

“Peptide segment” or “peptide domain” refers to a portion or fragment of a larger polypeptide or protein. A peptide segment or domain need not on its own have functional activity, although in some instances, a peptide segment or domain may correspond to a segment or domain of a polypeptide wherein the segment or domain has its own biological activity. A stability-associated peptide segment or domain is a peptide segment or domain found in a polypeptide that promotes stability, function, or folding compared to a related polypeptide lacking the peptide segment. A destabilizing-associated peptide segment is a peptide segment that is identified as causing a loss of stability, function or folding when present in a polypeptide. For example, B1P1, B6P3, B7P3 and B8P2 are segments/domains that promote thermostability in a chimeric polypeptide of the disclosure. In some embodiments, for example, a chimera has at least 1, 2, 3, or 4 thermostabilizing segments. For example, the disclosure provides chimeras that comprise at least 8 domains (i.e., B1-B2-B3-B4-B5-B6-B7-B8) comprising 1, 2, 3 or 4 domains comprising sequences that are at least 80-100% identical to a sequence selected from the group consisting of amino acid residue from about 1 to about x₁ of SEQ ID NO:2; from about amino acid residue x₅ to about x₆ of SEQ ID Nb:6; about amino acid residue x₆ to about x₇ of SEQ ID NO:6; and about amino acid residue x₇ to about x₈ of SEQ ID NO:4; wherein: x₁ is residue 43, 44, 45, 46, or 47 of SEQ ID NO:2, x₅ is residue 216, 217, 218, 219, 220, 221, 222 or 223 of SEQ ID NO:6; x₆ is residue 253, 254, 255, 256, 257, 258, 259 or 260 of SEQ ID NO:6; x₇ is residue 309, 310, 311, 312, 313, 314, 315 or 318 of SEQ ID NO:4 or SEQ ID NO:6; and x₈ is an amino acid residue corresponding to the C-terminus of the polypeptide having the sequence of SEQ ID NO:4.

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Fused,” “operably linked,” and “operably associated” are used interchangeably herein to broadly refer to a chemical or physical coupling of two otherwise distinct domains or peptide segments, wherein each domain or peptide segment when operably linked can provide a functional polypeptide having a desired activity. Domains or peptide segments can be directly linked or connected through peptide linkers such that they are functional or can be fused through other intermediates or chemical bonds. For example, two domains can be part of the same coding sequence, wherein the polynucleotides are in frame such that the polynucleotide when transcribed encodes a single mRNA that when translated comprises both domains as a single polypeptide. Alternatively, both domains can be separately expressed as individual polypeptides and fused to one another using chemical methods. Typically, the coding domains will be linked “in-frame” either directly of separated by a peptide linker and encoded by a single polynucleotide. Various coding sequences for peptide linkers and peptide are known in the art.

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the disclosure can be ribonucleotides, deoxyribonucleotides, or modified forms of either nucleotide. A polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA.

“Nucleic acid segment,” “oligonucleotide segment” or “polynucleotide segment” refers to a portion of a larger polynucleotide molecule. The polynucleotide segment need not correspond to an encoded functional domain of a protein; however, in some instances the segment will encode a functional domain of a protein. A polynucleotide segment can be about 6 nucleotides or more in length (e.g., 6-20, 20-50, 50-100, 100-200, 200-300, 300-400 or more nucleotides in length). A stability-associated peptide segment can be encoded by a stability-associated polynucleotide segment, wherein the peptide segment promotes stability, function, or folding compared to a polypeptide lacking the peptide segment.

“Chimera” refers to a combination of at least two segments or domains of at least two different parent proteins or polypeptides. As appreciated by one of skill in the art, the segments need not actually come from each of the parents, as it is the particular sequence that is relevant, and not the physical nucleic acids themselves. For example, a chimeric fungal class II cellobiohydrolases (CBH II cellulases) will have at least two segments from two different parent CBH II polypeptides. The two segments are connected so as to result in a new polypeptide having cellobiohydrolase activity. In other words, a protein will not be a chimera if it has the identical sequence of either one of the full length parents. A chimeric polypeptide can comprise more than two segments from two different parent proteins. For example, there may be 2, 3, 4, 5-10, 10-20, or more parents for each final chimera or library of chimeras. The segment of each parent polypeptide can be very short or very long, the segments can range in length of contiguous amino acids from 1 to about 90%, 95%, 98%, or 99% of the entire length of the protein. In one embodiment, the minimum length is 10 amino acids. In one embodiment, a single crossover point is defined for two parents. The crossover location defines where one parent's amino acid segment will stop and where the next parent's amino acid segment will start. Thus, a simple chimera would only have one crossover location where the segment before that crossover location would belong to a first parent and the segment after that crossover location would belong to a second parent. In one embodiment, the chimera has more than one crossover location. For example, 2, 3, 4, 5, 6, 7, 8, 9, 10, 11-30, or more crossover locations. How these crossover locations are named and defined are both discussed below. In an embodiment where there are two crossover locations and two parents, there will be a first contiguous segment from a first parent, followed by a second contiguous segment from a second parent, followed by a third contiguous segment from the first parent or yet a different parent. Contiguous is meant to denote that there is nothing of significance interrupting the segments. These contiguous segments are connected to form a contiguous amino acid sequence. For example, a CBH II chimera from Humicola insolens (hereinafter “1”) and H. jecori (hereinafter “2”), with two crossovers at 100 and 150, could have the first 100 amino acids from 1, followed by the next 50 from 2, followed by the remainder of the amino acids from 1, all connected in one contiguous amino acid chain. Alternatively, the CBH II chimera could have the first 100 amino acids from 2, the next 50 from 1 and the remainder followed by 2. As appreciated by one of skill in the art, variants of chimeras exist as well as the exact sequences. Thus, not 100% of each segment need be present in the final chimera if it is a variant chimera. The amount that may be altered, either through additional residues or removal or alteration of residues will be defined as the term variant is defined. Of course, as understood by one of skill in the art, the above discussion applies not only to amino acids but also nucleic acids which encode for the amino acids.

“Conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acids having aromatic side chains is substituted with another amino acid having an aromatic side chain, e.g., phenylalanine, tyrosine, tryptophan, and histidine; an amino acid with a basic side chain is substituted with another amino acid with a basis side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Isolated polypeptide” refers to a polypeptide which is separated from other contaminants that naturally accompany it, e.g., protein, lipids, and polynucleotides. The term embraces polypeptides which have been removed or purified from their naturally-occurring environment or expression system (e.g., host cell or in vitro synthesis).

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

“Reference sequence” refers to a defined sequence used as a basis for a sequence comparison. A reference sequence may be a subset of a larger sequence, for example, a segment of a full-length gene or polypeptide sequence. Generally, a reference sequence can be at least 20 nucleotide or amino acid residues in length, at least 25 nucleotide or residues in length, at least 50 nucleotides or residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

“Sequence identity” means that two amino acid sequences are substantially identical (e.g., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described elsewhere herein) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

“Functional” refers to a polypeptide which possesses either the native biological activity of the naturally-produced' proteins of its type, or any specific desired activity, for example as judged by its ability to bind to ligand molecules or carry out an enzymatic reaction.

The disclosure describes a directed SCHEMA recombination library to generate cellobiohydrolase enzymes based on a particularly members of this enzyme family, and more particularly cellobiohydrolase II enzymes (e.g., H. insolens is parent “1” (SEQ ID NO:2), H. jecorina is parent “2” (SEQ ID NO:4) and C. thermophilum is parent “3” (SEQ ID NO:6)). SCHEMA is a computational based method for predicting which fragments of related proteins can be recombined without affecting the structural integrity of the protein (see, e.g., Meyer et al., (2003) Protein Sci., 12:1686-1693). This computational approached identified seven recombination points in the CBH II parental proteins, thereby allowing the formation of a library of CBH II chimera polypeptides, where each polypeptide comprise eight segments. Chimeras with higher stability are identifiable by determining the additive contribution of each segment to the overall stability, either by use of linear regression of sequence-stability data, or by reliance on consensus analysis of the MSAs of folded versus unfolded proteins. SCHEMA recombination ensures that the chimeras retain biological function and exhibit high sequence diversity by conserving important functional residues while exchanging tolerant ones.

Thus, as illustrated by various embodiments herein, the disclosure provides CBH II polypeptides comprising a chimera of parental domains. In some embodiments, the polypeptide comprises a chimera having a plurality of domains from N- to C-terminus from different parental CBH II proteins: (segment 1)-(segment 2)-(segment 3)-(segment 4)-(segment 5)-(segment 6)-(segment 7)-(segment 8);

wherein segment 1 comprises amino acid residue from about 1 to about x₁ of SEQ ID NO:2 (“1”), SEQ ID NO:4 (“2”) or SEQ ID NO:6 (“3”); segment 2 is from about amino acid residue x₁ to about x₂ of SEQ ID NO:2 (“1”), SEQ ID NO:4 (“2”) or SEQ ID NO:6 (“3”); segment 3 is from about amino acid residue x₂ to about x₃ of SEQ ID NO:2 (“1”), SEQ ID NO:4 (“2”) or SEQ ID NO:6 (“3”); segment 4 is from about amino acid residue x₃ to about x₄ of SEQ ID NO:2 (“1”), SEQ ID NO:4 (“2”) or SEQ ID NO:6 (“3”); segment 5 is from about amino acid residue x₄ to about x₅ of SEQ ID NO:2 (“1”), SEQ ID NO:4 (“2”) or SEQ ID NO:6 (“3”); segment 6 is from about amino acid residue x₅ to about x₆ of SEQ ID NO:2 (“1”), SEQ ID NO:4 (“2”) or SEQ ID NO:6 (“3”); segment 7 is from about amino acid residue x₆ to about x₇ of SEQ ID NO:2 (“1”), SEQ ID NO:4 (“2”) or SEQ ID NO:6 (“3”); and segment 8 is from about amino acid residue x₇ to about x₈ of SEQ ID NO:2 (“1”), SEQ ID NO:4 (“2”) or SEQ ID NO:6 (“3”);

wherein: x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6.

Using the foregoing domain references a number of chimeric structure were generated as set forth in Table 1.

TABLE 1
1,588 CBH II chimera sequences with T₅₀ values predicted to be greater
than the measured T₅₀ value of 64.8 C for the H. insolens parent CBH II.

| | | | | | | | |
|---|---|---|---|---|---|---|---|
| 31313232 | 13132231 | 13212231 | 21113231 | 22112331 | 33211132 | 22223232 | 32123131 |
| 31323333 | 11221233 | 13331133 | 21133232 | 21222133 | 33211131 | 31213132 | 11221333 |
| 11212133 | 33123232 | 13232232 | 22221133 | 32333132 | 12311232 | 22223231 | 11211231 |
| 31313231 | 33123231 | 13232231 | 21133231 | 32333131 | 12321333 | 22322232 | 11231232 |
| 31333232 | 21311333 | 22121133 | 23211232 | 22132332 | 33231132 | 31213131 | 11231231 |
| 11232133 | 21331333 | 33223232 | 23221333 | 22132331 | 12311231 | 31312132 | 33123332 |
| 31333231 | 32213332 | 23111232 | 23211231 | 33313332 | 33231131 | 22322231 | 33123331 |
| 21323112 | 32213331 | 23121333 | 23231232 | 33313331 | 12331232 | 31312131 | 31321232 |
| 21323111 | 32312332 | 33223231 | 11311333 | 33333332 | 12331231 | 31233132 | 31321231 |
| 32113332 | 21211133 | 33322232 | 23231231 | 33333331 | 23122232 | 31233131 | 33122132 |
| 32113331 | 32312331 | 23111231 | 21122133 | 33213132 | 23122231 | 31332132 | 33122131 |
| 31223133 | 13321112 | 33322231 | 11331333 | 33213131 | 11113232 | 31332131 | 12123232 |
| 21111133 | 32233332 | 23131232 | 11211133 | 33312132 | 11123333 | 23321133 | 12123231 |
| 31322133 | 13321111 | 23131231 | 11231133 | 33312131 | 11113231 | 23222232 | 32121332 |
| 32133332 | 32233331 | 11323112 | 33113132 | 12313232 | 11133232 | 23222231 | 32121331 |
| 32133331 | 32332332 | 11323111 | 33113131 | 12323333 | 12221133 | 11213232 | 21323133 |
| 21131133 | 21231133 | 11111133 | 33133132 | 33233132 | 11133231 | 11223333 | 13122232 |
| 32112132 | 32332331 | 11131133 | 33133131 | 12313231 | 31111332 | 11213231 | 13122231 |
| 32112131 | 32121232 | 32221232 | 31321133 | 33233131 | 31111331 | 11312232 | 22113132 |
| 32132132 | 32121231 | 32221231 | 31222232 | 33332132 | 31131332 | 11322333 | 22113131 |
| 32132131 | 13313133 | 22313232 | 31222231 | 33332131 | 13211232 | 11312231 | 22133132 |
| 21321312 | 32212132 | 22323333 | 12123133 | 12333232 | 31131331 | 11233232 | 22133131 |
| 33112332 | 32212131 | 22313231 | 32111132 | 12333231 | 13221333 | 11233231 | 23113332 |
| 21321311 | 13333133 | 22333232 | 13113232 | 32311332 | 13211231 | 11332232 | 23113331 |
| 33112331 | 32232132 | 22333231 | 13123333 | 32311331 | 13231232 | 11332231 | 23133332 |
| 11223233 | 32232131 | 31122232 | 32111131 | 32331332 | 22212332 | 31211332 | 23133331 |
| 33132332 | 33212332 | 31122231 | 13113231 | 32331331 | 13231231 | 31211331 | 12212332 |
| 11322233 | 33212331 | 11321312 | 32131132 | 12223133 | 22212331 | 31231332 | 21311232 |
| 33132331 | 22123133 | 11321311 | 13133232 | 12322133 | 11122133 | 31231331 | 12212331 |
| 31211232 | 33232332 | 22223133 | 32131131 | 31113332 | 22232332 | 12112332 | 21321333 |
| 31221333 | 33232331 | 22322133 | 13133231 | 31113331 | 22232331 | 31323232 | 21311231 |
| 31211231 | 23113232 | 23213232 | 33111332 | 31133332 | 33321232 | 12112331 | 12232332 |
| 21313333 | 23123333 | 23223333 | 33111331 | 32211132 | 33321231 | 11222133 | 21331232 |
| 31231232 | 23113231 | 23213231 | 33131332 | 13213232 | 23323133 | 31323231 | 12232331 |
| 31231231 | 23133232 | 23312232 | 11321233 | 31133331 | 31213332 | 12132332 | 21331231 |
| 21221112 | 23133231 | 23322333 | 33131331 | 13223333 | 31213331 | 12132331 | 23112132 |
| 21333333 | 11121112 | 23312231 | 13122133 | 32211131 | 31312332 | 32123332 | 23112131 |
| 21221111 | 12311133 | 23233232 | 12111232 | 13213231 | 31312331 | 32123331 | 23132132 |
| 12112232 | 11121111 | 11313333 | 12121333 | 13312232 | 31233332 | 21121133 | 23132131 |
| 12122333 | 12212232 | 23233231 | 12111231 | 13322333 | 31233331 | 32122132 | 32223332 |
| 12112231 | 12222333 | 23332232 | 12131232 | 13312231 | 31332332 | 32122131 | 22111332 |
| 12132232 | 12212231 | 23332231 | 12131231 | 32231132 | 31332331 | 33122332 | 32223331 |
| 12132231 | 21321233 | 11221112 | 32313332 | 13233232 | 31121232 | 33122331 | 32322332 |
| 21213133 | 12331133 | 11333333 | 32313331 | 32231131 | 31121231 | 31221232 | 22111331 |
| 21312133 | 12232232 | 11221111 | 21311133 | 13233231 | 22321133 | 31221231 | 21221133 |
| 13323112 | 12232231 | 23222133 | 21212232 | 13332232 | 22222232 | 21313232 | 32322331 |
| 13323111 | 13311333 | 11213133 | 21222333 | 13332231 | 31212132 | 21323333 | 22131332 |
| 21233133 | 23122133 | 11312133 | 32333332 | 33211332 | 22222231 | 21313231 | 22131331 |
| 21332133 | 13331333 | 11233133 | 21212231 | 33211331 | 31212131 | 21333232 | 13323133 |
| 32123232 | 11113133 | 11332133 | 32333331 | 33231332 | 31232132 | 21333231 | 32222132 |
| 32123231 | 11133133 | 22211232 | 21331133 | 33231331 | 31232131 | 12122232 | 32222131 |
| 13111133 | 32223232 | 22221333 | 21232232 | 22122232 | 23311232 | 12122231 | 33311132 |
| 13131133 | 22111232 | 22211231 | 21232231 | 31112132 | 23321333 | 22113332 | 33311131 |
| 12321112 | 22121333 | 22231232 | 32213132 | 22122231 | 23311231 | 22113331 | 33222332 |
| 12321111 | 32223231 | 22231231 | 32213131 | 31112131 | 23331232 | 21223133 | 33222331 |
| 33122232 | 32322232 | 31323133 | 32312132 | 31132132 | 23331231 | 21322133 | 33331132 |
| 33122231 | 22111231 | 21112232 | 32312131 | 33323232 | 21123133 | 22133332 | 33331131 |
| 21211333 | 32322231 | 21122333 | 32233132 | 31132131 | 23221133 | 22133331 | 23123232 |
| 13321312 | 22131232 | 21112231 | 32233131 | 13222133 | 11311133 | 13121133 | 23123231 |
| 13321311 | 22131231 | 21132232 | 32332132 | 33323231 | 11212232 | 22112132 | 12321133 |
| 21231333 | 13211133 | 21132231 | 32332131 | 12211232 | 11222333 | 22112131 | 12222232 |
| 11123112 | 13231133 | 32113132 | 33213332 | 12221333 | 11212231 | 22132132 | 12222231 |
| 12313133 | 11323312 | 32113131 | 33213331 | 12211231 | 11331133 | 22132131 | 13311232 |
| 11123111 | 11323311 | 11211333 | 33312332 | 12231232 | 11232232 | 33313132 | 13321333 |
| 21323233 | 33321133 | 32133132 | 33312331 | 12231231 | 11232231 | 33313131 | 13311231 |
| 12333133 | 33222232 | 32133131 | 33233332 | 23121133 | 31223232 | 23112332 | 22213332 |
| 13313333 | 33222231 | 11231333 | 33233331 | 11112232 | 21111232 | 23112331 | 13331232 |
| 32212332 | 11111333 | 33113332 | 33332332 | 11122333 | 21121333 | 33333132 | 22213331 |
| 32212331 | 11131333 | 33113331 | 33332331 | 11112231 | 31223231 | 33333131 | 22312332 |
| 13221112 | 11223112 | 33133332 | 33121232 | 11132232 | 31322232 | 23132332 | 13331231 |
| 13333333 | 11223111 | 11323233 | 33121231 | 32321232 | 21111231 | 23132331 | 22312331 |
| 13221111 | 11322112 | 33133331 | 33212132 | 11132231 | 31322231 | 21211232 | 11123133 |
| 32232332 | 11322111 | 31311232 | 33212131 | 32321231 | 21131232 | 21221333 | 22233332 |
| 32232331 | 13321233 | 31321333 | 12213232 | 31123232 | 21131231 | 21211231 | 22233331 |
| 22113232 | 22213232 | 31311231 | 12223333 | 31123231 | 32122332 | 21231232 | 22332332 |
| 22123333 | 22223333 | 31331232 | 12213231 | 22323133 | 32122331 | 21231231 | 22332331 |
| 22113231 | 22213231 | 31331231 | 12312232 | 33221232 | 13123133 | 12323133 | 22121232 |
| 22133232 | 22312232 | 21321112 | 12322333 | 33221231 | 33111132 | 32311132 | 31111132 |
| 22133231 | 22322333 | 33112132 | 33232132 | 23313232 | 33111131 | 13313232 | 22121231 |
| 13213133 | 22312231 | 21321111 | 12312231 | 23323333 | 33131132 | 13323333 | 31111131 |
| 13312133 | 22233232 | 33112131 | 33232131 | 23313231 | 33131131 | 32311131 | 31131132 |
| 13233133 | 22233231 | 12113232 | 12233232 | 23333232 | 21213232 | 13313231 | 31131131 |
| 13332133 | 22332232 | 12123333 | 12233231 | 23333231 | 21223333 | 32222332 | 13221133 |
| 11121312 | 22332231 | 12113231 | 12332232 | 11321112 | 21213231 | 32222331 | 22212132 |
| 12311333 | 31121133 | 33132132 | 12332231 | 11321111 | 21312232 | 32331132 | 22212131 |
| 11121311 | 11221312 | 33132131 | 32211332 | 23223133 | 21322333 | 13333232 | 22232132 |
| 22122133 | 11221311 | 12133232 | 32211331 | 23322133 | 21312231 | 32331131 | 22232131 |
| 12331333 | 22222133 | 12133231 | 23123133 | 11313133 | 21233232 | 13333231 | 23212332 |
| 33323133 | 23311133 | 32111332 | 32231332 | 11333133 | 21233231 | 33311332 | 23212331 |
| 23112232 | 23212232 | 32111331 | 32231331 | 22311232 | 21332232 | 33311331 | 23232332 |
| 23122333 | 23222333 | 31221133 | 32323232 | 22321333 | 21332231 | 33331332 | 23232331 |
| 23112231 | 23212231 | 32131332 | 12222133 | 22311231 | 12121133 | 22123232 | 11111232 |
| 11113333 | 23331133 | 21313133 | 32323231 | 31212332 | 13111232 | 33331331 | 11121333 |
| 23132232 | 11213333 | 32131331 | 31112332 | 31212331 | 13121333 | 31113132 | 11111231 |
| 23132231 | 23232232 | 21333133 | 31112331 | 22331232 | 13111231 | 22123231 | 11131232 |
| 11133333 | 11312333 | 12122133 | 13311133 | 22331231 | 32313132 | 31113131 | 11131231 |
| 12211133 | 23232231 | 13112232 | 31132332 | 31232332 | 32313131 | 31133132 | 31313332 |
| 21221233 | 11233333 | 13122333 | 13212232 | 31232331 | 13131232 | 31133131 | 31313331 |
| 12231133 | 11332333 | 13112231 | 31132331 | 21113232 | 22112332 | 13223133 | 31333332 |
| 13211333 | 11121233 | 13132232 | 13222333 | 21123333 | 13131231 | 13322133 | 31333331 |
| 22131131 | 22331331 | 13111331 | 33323132 | 33321132 | 22121132 | 11311132 | 11321132 |
| 33223332 | 21113332 | 13131332 | 33323131 | 33321131 | 22121131 | 11311131 | 11321131 |
| 23111332 | 21113331 | 13131331 | 23122332 | 11111332 | 23121332 | 11222332 | 11323132 |
| 33223331 | 21133332 | 21212132 | 23122331 | 11111331 | 23121331 | 11222331 | 11323131 |
| 33322332 | 22211132 | 21212131 | 11113332 | 11131332 | 11111132 | 11331132 | 11321332 |
| 23111331 | 21133331 | 21232132 | 11113331 | 11131331 | 11111131 | 11331131 | 11321331 |
| 33322331 | 22211131 | 21232131 | 11133332 | 13321232 | 11131132 | 21121332 | 11221132 |
| 23131332 | 22231132 | 12313332 | 12211132 | 13321231 | 11131131 | 21121331 | 11221131 |
| 23131331 | 22231131 | 12313331 | 11133331 | 22223332 | 22323332 | 13123132 | 21321132 |
| 33222132 | 23211332 | 12333332 | 12211131 | 22223331 | 22323331 | 13123131 | 21321131 |
| 33222131 | 23211331 | 12333331 | 21221232 | 22322332 | 22223132 | 21223332 | 13321132 |
| 12223232 | 23231332 | 33121132 | 21221231 | 22322331 | 22223131 | 21223331 | 13321131 |
| 12223231 | 23231331 | 33121131 | 12231132 | 31121132 | 22322132 | 21322332 | 11121132 |
| 12322232 | 21112132 | 12213132 | 12231131 | 31121131 | 22322131 | 21322331 | 11121131 |
| 12322231 | 21112131 | 12213131 | 13211332 | 22222132 | 23223332 | 12121132 | 11323332 |
| 32221332 | 21132132 | 12312132 | 13211331 | 22222131 | 23223331 | 12121131 | 11323331 |
| 32221331 | 23323232 | 12312131 | 13231332 | 23311132 | 23322332 | 13121332 | 11223132 |
| 22313332 | 21132131 | 21223232 | 13231331 | 23311131 | 23322331 | 13121331 | 11223131 |
| 22313331 | 23323231 | 21223231 | 11112132 | 23222332 | 11313332 | 21222132 | 11322132 |
| 22333332 | 11323133 | 21322232 | 11112131 | 23222331 | 11313331 | 21222131 | 11322131 |
| 22333331 | 22321232 | 12233132 | 11132132 | 23331132 | 11333332 | 12323332 | 11221332 |
| 31122332 | 31311132 | 21322231 | 32321132 | 11213332 | 11333331 | 12323331 | 11221331 |
| 31122331 | 22321231 | 12233131 | 13323232 | 23331131 | 23222132 | 12223132 | 21323132 |
| 13321133 | 31311131 | 12332132 | 11132131 | 11213331 | 23222131 | 12223131 | 21323131 |
| 13222232 | 31222332 | 12332131 | 32321131 | 11312332 | 11213132 | 12322132 | 21321332 |
| 13222231 | 31222331 | 13213332 | 13323231 | 11312331 | 11213131 | 12322131 | 21321331 |
| 22213132 | 31331132 | 13213331 | 33321332 | 11233332 | 11312132 | 13223332 | 21221132 |
| 22213131 | 31331131 | 13312332 | 33321331 | 11233331 | 11312131 | 13223331 | 21221131 |
| 22312132 | 12113132 | 13312331 | 31123132 | 11332332 | 11233132 | 13322332 | 13323132 |
| 22312131 | 12113131 | 13233332 | 31123131 | 11332331 | 11233131 | 13322331 | 13323131 |
| 22233132 | 21123232 | 13233331 | 33221132 | 11121232 | 11332132 | 13222132 | 12321132 |
| 22233131 | 21123231 | 13332332 | 33221131 | 11121231 | 11332131 | 13222131 | 12321131 |
| 22332132 | 12133132 | 13332331 | 23313132 | 31323332 | 22221332 | 12221332 | 13321332 |
| 22332131 | 12133131 | 13121232 | 12321232 | 11212132 | 22221331 | 12221331 | 13321331 |
| 23213332 | 13113332 | 13121231 | 23313131 | 31323331 | 31323132 | 23121132 | 11123132 |
| 23213331 | 13113331 | 32323132 | 12321231 | 11212131 | 31323131 | 23121131 | 11123131 |
| 23312332 | 13133332 | 32323131 | 23333132 | 11232132 | 21122332 | 11122332 | 13221132 |
| 23312331 | 13133331 | 22122332 | 23333131 | 11232131 | 21122331 | 11122331 | 13221131 |
| 23233332 | 23221232 | 22122331 | 11123232 | 31223132 | 11211332 | 22323132 | 11121332 |
| 23233331 | 23221231 | 33323332 | 11123231 | 21111132 | 11211331 | 22323131 | 11121331 |
| 23332332 | 11311232 | 13212132 | 31121332 | 31223131 | 11231332 | 23323332 | 23321132 |
| 23332331 | 11321333 | 33323331 | 31121331 | 31322132 | 11231331 | 23323331 | 23321131 |
| 23121232 | 11311231 | 13212131 | 13221232 | 21111131 | 11323232 | 23223132 | 11223332 |
| 23121231 | 11331232 | 13232132 | 13221231 | 31322131 | 11323231 | 23223131 | 11223331 |
| 23212132 | 11331231 | 13232131 | 22311132 | 21131132 | 31321332 | 23322132 | 11322332 |
| 23212131 | 13112132 | 12211332 | 22311131 | 21131131 | 31321331 | 23322131 | 11322331 |
| 23232132 | 13112131 | 12211331 | 22222332 | 11223232 | 12123332 | 11313132 | 11222132 |
| 23232131 | 13132132 | 12231332 | 22222331 | 11223231 | 12123331 | 11313131 | 11222131 |
| 22211332 | 13132131 | 12231331 | 22331132 | 11322232 | 31221132 | 11333132 | 21121132 |
| 22211331 | 12111332 | 33223132 | 22331131 | 11322231 | 31221131 | 11333131 | 21121131 |
| 11121133 | 12111331 | 23111132 | 23311332 | 31221332 | 21313132 | 22321332 | 21323332 |
| 22231332 | 11221133 | 33223131 | 23311331 | 31221331 | 21313131 | 22321331 | 21323331 |
| 22231331 | 12131332 | 33322132 | 23331332 | 21313332 | 21333132 | 21123332 | 21223132 |
| 22323232 | 12131331 | 23111131 | 23331331 | 21313331 | 21333131 | 21123331 | 21223131 |
| 31313132 | 33123132 | 33322131 | 21113132 | 21333332 | 12122132 | 22221132 | 21322132 |
| 22323231 | 33123131 | 12323232 | 21113131 | 21333331 | 12122131 | 22221131 | 21322131 |
| 31313131 | 21212332 | 12323231 | 21133132 | 12122332 | 13122332 | 23221332 | 13121132 |
| 21112332 | 21212331 | 23131132 | 21133131 | 12122331 | 13122331 | 23221331 | 13121131 |
| 21112331 | 21232332 | 23131131 | 23211132 | 21213132 | 11221232 | 11311332 | 21221332 |
| 31333132 | 21232331 | 11112332 | 23211131 | 21213131 | 11221231 | 11311331 | 21221331 |
| 31333131 | 32121132 | 11112331 | 23231132 | 21312132 | 21311332 | 21122132 | 12323132 |
| 21132332 | 13123232 | 11132332 | 23231131 | 21312131 | 21311331 | 21122131 | 12323131 |
| 21132331 | 32121131 | 32321332 | 11212332 | 21233132 | 21331332 | 11331332 | 13323332 |
| 23223232 | 13123231 | 11132331 | 11212331 | 21233131 | 21331331 | 11331331 | 13323331 |
| 23223231 | 33121332 | 32321331 | 11232332 | 21332132 | 21211132 | 11211132 | 13223132 |
| 23322232 | 33121331 | 31123332 | 11232331 | 21332131 | 21211131 | 11211131 | 13223131 |
| 23322231 | 12213332 | 31123331 | 31223332 | 13111132 | 21231132 | 11231132 | 13322132 |
| 11313232 | 12213331 | 32221132 | 21111332 | 13111131 | 21231131 | 11231131 | 13322131 |
| 11323333 | 12312332 | 13223232 | 31223331 | 13131132 | 13313132 | 31321132 | 12321332 |
| 11313231 | 12312331 | 32221131 | 31322332 | 13131131 | 13313131 | 31321131 | 12321331 |
| 11333232 | 12233332 | 13223231 | 21111331 | 21211332 | 13333132 | 12123132 | 11123332 |
| 11333231 | 12233331 | 13322232 | 31322331 | 21211331 | 13333131 | 12123131 | 11123331 |
| 31311332 | 12332332 | 13322231 | 21131332 | 21231332 | 22123132 | 13123332 | 12221132 |
| 31311331 | 12332331 | 22313132 | 21131331 | 21231331 | 22123131 | 13123331 | 12221131 |
| 31331332 | 23113132 | 22313131 | 31222132 | 12313132 | 23123332 | 11321232 | 13221332 |
| 31331331 | 12121232 | 22333132 | 31222131 | 12313131 | 23123331 | 11321231 | 13221331 |
| 12113332 | 23113131 | 33221332 | 23321232 | 21323232 | 12311132 | 13122132 | 11122132 |
| 12113331 | 12121231 | 22333131 | 23321231 | 21323231 | 12311131 | 13122131 | 11122131 |
| 11223133 | 23133132 | 33221331 | 13113132 | 12333132 | 12222332 | 12121332 | 23323132 |
| 11322133 | 23133131 | 23313332 | 13113131 | 12333131 | 21321232 | 12121331 | 23323131 |
| 12133332 | 32323332 | 23313331 | 13133132 | 13313332 | 12222331 | 21311132 | 22321132 |
| 12133331 | 12212132 | 31122132 | 13133131 | 13313331 | 21321231 | 21311131 | 22321131 |
| 22221232 | 32323331 | 23333332 | 11321133 | 13333332 | 12331132 | 21222332 | 23321332 |
| 31211132 | 12212131 | 31122131 | 11222232 | 13333331 | 12331131 | 21222331 | 23321331 |
| 22221231 | 21321133 | 23333331 | 11222231 | 22123332 | 13311332 | 21331132 | 21123132 |
| 31211131 | 21222232 | 23213132 | 21213332 | 22123331 | 13311331 | 21331131 | 21123131 |
| 31231132 | 21222231 | 12221232 | 21213331 | 13213132 | 23122132 | 12223332 | 23221132 |
| 31231131 | 12232132 | 23213131 | 21312332 | 13213131 | 23122131 | 12223331 | 23221131 |
| 12112132 | 12232131 | 23312132 | 21312331 | 13312132 | 13331332 | 12322332 | |
| 12112131 | 13212332 | 12221231 | 21233332 | 13312131 | 13331331 | 12322331 | |
| 21122232 | 13212331 | 23312131 | 21233331 | 13233132 | 11113132 | 23123132 | |
| 21122231 | 13232332 | 23233132 | 21332332 | 13233131 | 11113131 | 23123131 | |
| 12132132 | 13232331 | 23233131 | 12111132 | 13332132 | 11133132 | 12222132 | |
| 12132131 | 32223132 | 23332132 | 21332331 | 13332131 | 11133131 | 12222131 | |
| 13112332 | 22111132 | 23332131 | 12111131 | 12311332 | 22121332 | 13311132 | |
| 13112331 | 32223131 | 22311332 | 21121232 | 12311331 | 22121331 | 13311131 | |
| 13132332 | 32322132 | 22311331 | 21121231 | 22122132 | 13211132 | 13222332 | |
| 13132331 | 22111131 | 11122232 | 12131132 | 22122131 | 13211131 | 13222331 | |
| 32123132 | 32322131 | 22331332 | 12131131 | 12331332 | 13231132 | 13331132 | |
| 11211232 | 22131132 | 11122231 | 13111332 | 12331331 | 13231131 | 13331131 | |

Referring to the table above, each digit refers to a domain of a chimeric CBH II polypeptide. The number denotes the parental strand of the domain. For example, a chimeric CBH II chimeric polypeptide having the sequence 12111131, indicates that the polypeptide comprises a sequence from the N-terminus to the C-terminus of: amino acids from about 1 to x₁ of SEQ ID NO:2 (“1”) linked to amino acids from about x₁ to x₂ of SEQ ID NO:4 (“2”) linked to amino acids from about x₂ to about x₃ of SEQ ID NO:2 linked to amino acids from about x₃ to about x₄ of SEQ ID NO:2 linked to amino acids from about x₄ to about x₅ of SEQ ID NO:2 linked to amino acids from about x₅ to about x₆ of SEQ ID NO:2 linked to amino acids from about x₆ to x₇ of SEQ ID NO:6 (“3”) linked to amino acids from about x₇ to x₈ (e.g., the C-terminus) of SEQ ID NO:2.

In some embodiments, the CBH II polypeptide has a chimeric segment structure selected from the group consisting of: 11113132, 21333331, 21311131, 22232132, 33133132, 33213332, 13333232, 12133333, 13231111, 11313121, 11332333, 12213111, 23311333, 13111313, 31311112, 23231222, 33123313, 22212231, 21223122, 21131311, 23233133, 31212111 and 32333113.

In some embodiments, the polypeptide has improved thermostability compared to a wild-type polypeptide of SEQ ID NO:2, 4, or 6. The activity of the polypeptide can be measured with any one or combination of substrates as described in the examples. As will be apparent to the skilled artisan, other compounds within the class of compounds exemplified by those discussed in the examples can be tested and used.

In some embodiments, the polypeptide can comprise various changes to the amino acid sequence with respect to a reference sequence. The changes can be a substitution, deletion, or insertion of one or more amino acids. Where the change is a substitution, the change can be a conservative or a non-conservative substitution. Accordingly a chimera may comprise a combination of conservative and non-conservative substitutions.

Thus, in some embodiments, the polypeptides can comprise a general structure from N-terminus to C-terminus: (segment 1)-(segment 2)-(segment 3)-(segment 4)-(segment 5)-(segment 6)-(segment 7)-(segment 8),

wherein segment 1 comprises amino acid residue from about 1 to about x₁ of SEQ ID NO:2 (“1”), SEQ ID NO:4 (“2”) or SEQ ID NO:6 (“3”) and having 1-10 conservative amino acid substitutions; segment 2 is from about amino acid residue x₁ to about x₂ of SEQ ID NO:2 (“1”), SEQ ID NO:4 (“2”) or SEQ ID NO:6 (“3”) and having about 1-10 conservative amino acid substitutions; segment 3 is from about amino acid residue x₂ to about x₃ of SEQ ID NO:2 (“1”), SEQ ID NO:4 (“2”) or SEQ ID NO:6 (“3”) and having about 1-10 conservative amino acid substitutions; segment 4 is from about amino acid residue x₃ to about x₄ of SEQ ID NO:2 (“1”), SEQ ID NO:4 (“2”) or SEQ ID NO:6 (“3”) and having about 1-10 conservative amino acid substitutions; segment 5 is from about amino acid residue x₄ to about x₅ of SEQ ID NO:2 (“1”), SEQ ID NO:4 (“2”) or SEQ ID NO:6 (“3”) and having about 1-10 conservative amino acid substitutions; segment 6 is from about amino acid residue x₅ to about x₆ of SEQ ID NO:2 (“1”), SEQ ID NO:4 (“2”) or SEQ ID NO:6 (“3”) and having about 1-10 conservative amino acid substitutions; segment 7 is from about amino acid residue x₆ to about x₇ of SEQ ID NO:2 (“1”), SEQ ID NO:4 (“2”) or SEQ ID NO:6 (“3”) and having about 1-10 conservative amino acid substitutions; and segment 8 is from about amino acid residue x₇ to about x₈ of SEQ ID NO:2 (“1”), SEQ ID NO:4 (“2”) or SEQ ID NO:6 (“3”) and having about 1-10 conservative amino acid substitutions;

wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6 and wherein the chimera has an algorithm as set forth in Table 1.

In some embodiments, the number of substitutions can be 2, 3, 4, 5, 6, 8, 9, or 10, or more amino acid substitutions (e.g., 10-20, 21-30, 31-40 and the like amino acid substitutions).

In some embodiments, the functional chimera polypeptides can have cellobiohydrolase activity along with increased thermostability, such as for a defined substrate discussed in the Examples, and also have a level of amino acid sequence identity to a reference cellobiohydrolase, or segments thereof. The reference enzyme or segment, can be that of a wild-type (e.g., naturally occurring) or an engineered enzyme. Thus, in some embodiments, the polypeptides of the disclosure can comprise a general structure from N-terminus to C-terminus:

wherein segment 1 comprises a sequence that is at least 50-100% identity to amino acid residue from about 1 to about x₁ of SEQ ID NO:2 (“1”), SEQ ID NO:4 (“2”) or SEQ ID NO:6 (“3”); segment 2 comprises a sequence that is at least 50-100% identity to amino acid residue x₁ to about x₂ of SEQ ID NO:2 (“1”), SEQ ID NO:4 (“2”) or SEQ ID NO:6 (“3”); segment 3 comprises a sequence that is at least 50-100% identity to amino acid residue x₂ to about x₃ of SEQ ID NO:2 (“1”), SEQ ID NO:4 (“2”) or SEQ ID NO:6 (“3”); segment 4 comprises a sequence that is at least 50-100% identity to amino acid residue x₃ to about x₄ of SEQ ID NO:2 (“1”), SEQ ID NO:4 (“2”) or SEQ ID NO:6 (“3”); segment 5 comprises a sequence that is at least 50-100% identity to about amino acid residue x₄ to about x₅ of SEQ ID NO:2 (“1”), SEQ ID NO:4 (“2”) or SEQ ID NO:6 (“3”); segment 6 comprises a sequence that is at least 50-100% identity to amino acid residue x₅ to about x₆ of SEQ ID NO:2 (“1”), SEQ ID NO:4 (“2”) or SEQ ID NO:6 (“3”); segment 7 comprises a sequence that is at least 50-100% identity to amino acid residue x₆ to about x₇ of SEQ ID NO:2 (“1”), SEQ ID NO:4 (“2”) or SEQ ID NO:6 (“3”); and segment 8 comprises a sequence that is at least 50-100% identity to amino acid residue x₇ to about x₈ of SEQ ID NO:2 (“1”), SEQ ID NO:4 (“2”) or SEQ ID NO:6 (“3”);

wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6 and wherein the chimera has an algorithm as set forth in Table 1.

In some embodiments, each segment of the chimeric polypeptide can have at least 60%, 70%, 80%, 90%, 95%, 96%, 97%, 98%, or 99% or more sequence identity as compared to the reference segment indicated for each of the (segment 1), (segment 2), (segment 3), (segment 4)-(segment 5), (segment 6), (segment 7), and (segment 8) of SEQ ID NO:2, SEQ ID NO:4, or SEQ ID NO:6.

In some embodiments, the polypeptide variants can have improved thermostability compared to the enzyme activity of the wild-type polypeptide of SEQ ID NO:2, 4, or 6.

The chimeric enzymes described herein may be prepared in various forms, such as lysates, crude extracts, or isolated preparations. The polypeptides can be dissolved in suitable solutions; formulated as powders, such as an acetone powder (with or without stabilizers); or be prepared as lyophilizates. In some embodiments, the polypeptide can be an isolated polypeptide.

In some embodiments, the polypeptides can be in the form of arrays. The enzymes may be in a soluble form, for example, as solutions in the wells of mircotitre plates, or immobilized onto a substrate. The substrate can be a solid substrate or a porous substrate (e.g, membrane), which can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

The disclosure also provides polynucleotides encoding the engineered CBH II polypeptides disclosed herein. The polynucleotides may be operatively linked to one or more heterologous regulatory or control sequences that control gene expression to create a recombinant polynucleotide capable of expressing the polypeptide. Expression constructs containing a heterologous polynucleotide encoding the CBH II chimera can be introduced into appropriate host cells to express the polypeptide.

Given the knowledge of specific sequences of the CBH II chimera enzymes (e.g., the segment structure of the chimeric CBH II), the polynucleotide sequences will be apparent form the amino acid sequence of the engineered CBH II chimera enzymes to one of skill in the art and with reference to the polypeptide sequences and nucleic acid sequence described herein. The knowledge of the codons corresponding to various amino acids coupled with the knowledge of the amino acid sequence of the polypeptides allows those skilled in the art to make different polynucleotides encoding the polypeptides of the disclosure. Thus, the disclosure contemplates each and every possible variation of the polynucleotides that could be made by selecting combinations based on possible codon choices, and all such variations are to be considered specifically disclosed for any of the polypeptides described herein.

In some embodiments, the polynucleotides encode the polypeptides described herein but have about 80% or more sequence identity, about 85% or more sequence identity, about 90% or more sequence identity, about 91% or more sequence identity, about 92% or more sequence identity, about 93% or more sequence identity, about 94% or more sequence identity, about 95% or more sequence identity, about 96% or more sequence identity, about 97% or more sequence identity, about 98% or more sequence identity, or about 99% or more sequence identity at the nucleotide level to a reference polynucleotide encoding the CBH II chimera polypeptides.

In some embodiments, the isolated polynucleotides encoding the polypeptides may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007.

In some embodiments, the polynucleotides are operatively linked to control sequences for the expression of the polynucleotides and/or polypeptides. In some embodiments, the control sequence may be an appropriate promoter sequence, which can be obtained from genes encoding extracellular or intracellular polypeptides, either homologous or heterologous to the host cell. For bacterial host cells, suitable promoters for directing transcription of the nucleic acid constructs of the present disclosure, include the promoters obtained from the E. coli lac operon, Bacillus subtilis xylA and xylB genes, Bacillus megatarium xylose utilization genes (e.g.,Rygus et al., (1991) Appl. Microbiol. Biotechnol. 35:594-599; Meinhardt et al., (1989) Appl. Microbiol. Biotechnol. 30:343-350), prokaryotic beta-lactamase gene (Villa-Kamaroff et al., (1978) Proc. Natl. Acad. Sci. USA 75: 3727-3731), as well as the tac promoter (DeBoer et al., (1983) Proc. Natl. Acad. Sci. USA 80: 21-25). Various suitable promoters are described in “Useful proteins from recombinant bacteria” in Scientific American, 1980, 242:74-94; and in Sambrook et al., supra.

In some embodiments, the control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

In some embodiments, the control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

In some embodiments, the control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region. Effective signal peptide coding regions for bacterial host cells can be the signal peptide coding regions obtained from the genes for Bacillus NClB 11837 maltogenic amylase, Bacillus stearothermophilus alpha-amylase, Bacillus lichenifonnis subtilisin, Bacillus lichenifonnis beta-lactamase, Bacillus stearothermophilus neutral proteases (nprT, nprS, nprM), and Bacillus subtilis prsA. Further signal peptides are described by Simonen and Palva, (1993) Microbiol Rev 57: 109-137.

The disclosure is further directed to a recombinant expression vector comprising a polynucleotide encoding the engineered CBH II chimera polypeptides, and one or more expression regulating regions such as a promoter and a terminator, a replication origin, etc., depending on the type of hosts into which they are to be introduced. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon, may be used.

In some embodiments, the expression vector of the disclosure contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus lichenifonnis, or markers, which confer antibiotic resistance such as ampicillin, kanamycin, chloramphenicol or tetracycline resistance. Other useful markers will be apparent to the skilled artisan.

In another embodiment, the disclosure provides a host cell comprising a polynucleotide encoding the CBH II chimera polypeptide, the polynucleotide being operatively linked to one or more control sequences for expression of the polypeptide in the host cell. Host cells for use in expressing the polypeptides encoded by the expression vectors of the disclosure are well known in the art and include, but are not limited to, bacterial cells, such as E. coli and Bacillus megaterium; eukaryotic cells, such as yeast cells, CHO cells and the like, insect cells such as Drosophila S2 and Spodoptera Sf9 cells; animal cells such as CHO, COS, BHK, 293, and Bowes melanoma cells; and plant cells. Other suitable host cells will be apparent to the skilled artisan. Appropriate culture mediums and growth conditions for the above-described host cells are well known in the art.

The CBH II chimera polypeptides of the disclosure can be made by using methods well known in the art. Polynucleotides can be synthesized by recombinant techniques, such as that provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007. Polynucleotides encoding the enzymes, or the primers for amplification can also be prepared by standard solid-phase methods, according to known synthetic methods, for example using phosphoramidite method described by Beaucage et al., (1981) Tet Lett 22:1859-69, or the method described by Matthes et al., (1984) EMBO J. 3:801-05, e.g., as it is typically practiced in automated synthetic methods. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others.

Engineered enzymes expressed in a host cell can be recovered from the cells and or the culture medium using any one or more of the well known techniques for protein purification, including, among others, lysozyme treatment, sonication, filtration, salting-out, ultra-centrifugation, chromatography, and affinity separation (e.g., substrate bound antibodies). Suitable solutions for lysing and the high efficiency extraction of proteins from bacteria, such as E. coli, are commercially available under the trade name CelLytic BTM from Sigma-Aldrich of St. Louis Mo.

Chromatographic techniques for isolation of the polypeptides include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

Descriptions of SCHEMA directed recombination and synthesis of chimeric polypeptides are described in the examples herein, as well as in Otey et al., (2006), PLoS Biol. 4(5):e112; Meyer et al., (2003) Protein Sci., 12:1686-1693; U.S. patent application Ser. No. 12/024,515, filed Feb. 1, 2008; and U.S. patent application Ser. No. 12/027,885, filed Feb. 7, 2008; such references incorporated herein by reference in their entirety.

As discussed above, the polypeptide can be used in a variety of applications, such as, among others, biofuel generation, cellulose breakdown and the like.

For example, in one embodiment, a method for processing cellulose is provided. The method includes culturing a recombinant microorganism as provided herein that expresses a chimeric polypeptide of the disclosure in the presence of a suitable cellulose substrate and under conditions suitable for the catalysis by the chimeric polypeptide of the cellulose.

In yet another embodiment, a substantially purified chimeric polypeptide of the disclosure is contacted with a cellulose substrate under conditions that allow for the chimeric polypeptide degrade the cellulose. In one embodiment, the conditions include temperatures from about 35-65° C.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q□-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

Appropriate culture conditions are conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

The following examples are meant to further explain, but not limited the foregoing disclosure or the appended claims.

### EXAMPLES

CBH II expression plasmid construction. Parent and chimeric genes encoding CBH II enzymes were cloned into yeast expression vector YEp352/PGK91-1-αss (FIG. 6). DNA sequences encoding parent and chimeric CBH II catalytic domains were designed with S. cerevisiae codon bias using GeneDesigner software (DNA2.0) and synthesized by DNA2.0. The CBH II catalytic domain genes were digested with XhoI and KpnI, ligated into the vector between the XhoI and KpnI sites and transformed into E. coli XL-1 Blue (Stratagene). CBH II genes were sequenced using primers: CBH2L (5′-GCTGAACGTGTCATCGGTTAC-3′ (SEQ ID NO:9) and RSQ3080 (5′-GCAACACCTGGCAATTCCTTACC-3′ (SEQ ID NO:10)). C-terminal His₆ parent and chimera CBH II constructs were made by amplifying the CBH II gene with forward primer CBH2LPCR (5′-GCTGAACGTGTCATCGTTACTTAG-3′ (SEQ ID NO:11)) and reverse primers complementary to the appropriate CBH II gene with His₆ overhangs and stop codons. PCR products were ligated, transformed and sequenced as above.

CBH II enzyme expression in S. cerevisiae. S. cerevisiae strain YDR483W BY4742 (Matα his3Δ1 leu2Δ0 lys2Δ0 ura3Δ0 ΔKRE2, ATCC No. 4014317) was made competent using the EZ Yeast II Transformation Kit (Zymo Research), transformed with plasmid DNA and plated on synthetic dropout -uracil agar. Colonies were picked into 5 mL overnight cultures of synthetic dextrose casamino acids (SDCAA) media (20 g/L dextrose, 6.7 g/L Difco yeast nitrogen base, 5 g/L Bacto casamino acids, 5.4 g/L Na₂HPO₄, 8.56 g/L NaH₂PO₄.H₂O) supplemented with 20 ug/mL tryptophan and grown overnight at 30° C., 250 rpm. 5 mL cultures were expanded into 40 mL SDCAA in 250 mL Tunair flasks (Shelton Scientific) and shaken at 30° C., 250 rpm for 48 hours. Cultures were centrifuged, and supernatants were concentrated to 500 uL, using an Amicon ultrafiltration cell fitted with 30-kDa PES membrane, for use in t_{1/2} assays. Concentrated supernatants were brought to 1 mM phenylmethylsulfonylfluoride and 0.02% NaN₃. His₆-tagged CBH II proteins were purified using Ni—NTA spin columns (Qiagen) per the manufacturer's protocol and the proteins exchanged into 50 mM sodium acetate, pH 4.8, using Zeba-Spin desalting columns (Pierce). Purified protein concentration was determined using Pierce Coomassie Plus protein reagent with BSA as standard. SDS-PAGE analysis was performed by loading either 20 uL of concentrated culture supernatant or approximately 5 ug of purified CBH II enzyme onto a 7.5% Tris-HCl gel (Biorad) and staining with SimplyBlue safe stain (Invitrogen). CBH II supernatants or purified proteins were treated with EndoH (New England Biolabs) for 1 hr at 37° C. per the manufacturer's instructions. CBH II enzyme activity in concentrated yeast culture supernatants was measured by adding 37.5 uL concentrated culture supernatant to 37.5 uL PASC and incubating for 2 hr at 50° C. Reducing sugar equivalents formed were determined via Nelson-Somogyi assay as described below.

Half-life, specific activity, pH-activity and long-time cellulose hydrolysis measurements. Phosphoric acid swollen cellulose (PASC) was prepared. To enhance CBH II enzyme activity on the substrate, PASC was pre-incubated at a concentration of 10 g/L with 10 mg/mL A. niger endoglucanase (Sigma) in 50 mM sodium acetate, pH 4.8 for 1 hr at 37° C. Endoglucanase was inactivated by heating to 95° C. for 15 minutes, PASC was washed twice with 50 mM acetate buffer and resuspended at 10 g/L in deionzed water.

CBH II enzyme t_{1/2}s were measured by adding concentrated CBH II expression culture supernatant to 50 mM sodium acetate, pH 4.8 at a concentration giving A₅₂₀ of 0.5 as measured in the Nelson-Somogyi reducing sugar assay after incubation with treated PASC as described below. 37.5 uL CBH II enzyme/buffer mixtures were inactivated in a water bath at 63° C. After inactivation, 37.5 uL endoglucanase-treated PASC was added and hydrolysis was carried out for 2 hr at 50° C. Reaction supernatants were filtered through Multiscreen HTS plates (Millipore). Nelson-Somogyi assay log(A₅₂₀) values, obtained using a SpectraMax microplate reader (Molecular Devices) corrected for background absorbance, were plotted versus time and CBH II enzyme half-lives obtained from linear regression using Microsoft Excel.

For specific activity measurements, purified CBH II enzyme was added to PASC to give a final reaction volume of 75 uL 25 mM sodium acetate, pH 4.8, with 5 g/L PASC and CBH II enzyme concentration of 3 mg enzyme/g PASC. Incubation proceeded for 2 hr in a 50° C. water bath and the reducing sugar concentration determined. For pH/activity profile measurements, purified CBH II enzyme was added at a concentration of 300 ug/g PASC in a 75 uL reaction volume. Reactions were buffered with 12.5 mM sodium citrate/12.5 mM sodium phosphate, run for 16 hr at 50° C. and reducing sugar determined. Long-time cellulose hydrolysis measurements were performed with 300 uL volumes of 1 g/L treated PASC in 100 mM sodium acetate, pH 4.8, 20 mM NaCl. Purified CBH II enzyme was added at 100 ug/g PASC and reactions carried out in water baths for 40 hr prior to reducing sugar determination.

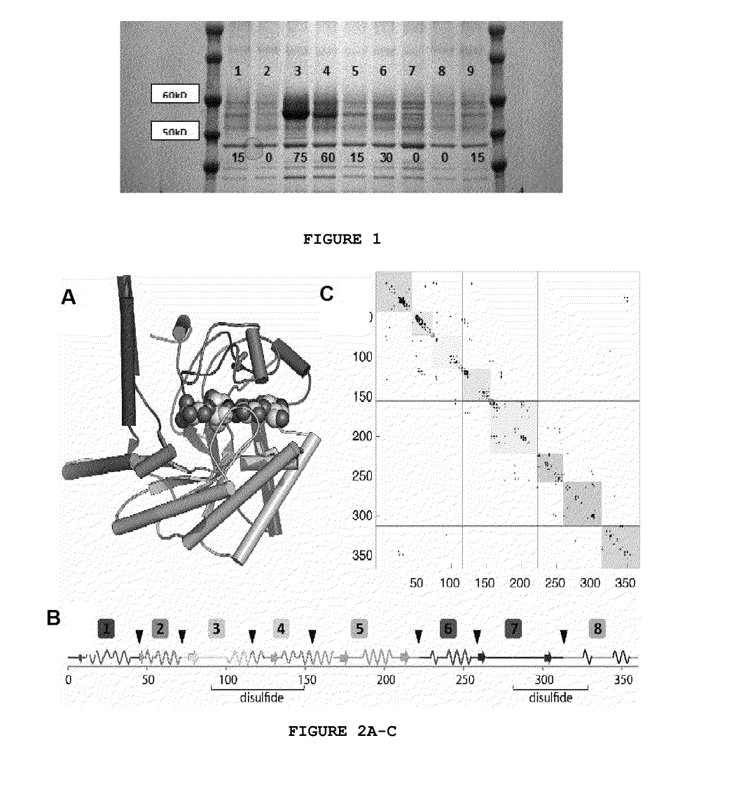

Five candidate parent genes encoding CBH II enzymes were synthesized with S. cerevisiae codon bias. All five contained identical N-terminal coding sequences, where residues 1-89 correspond to the cellulose binding module (CBM), flexible linker region and the five N-terminal residues of the H. jecorina catalytic domain. Two of the candidate CBH II enzymes, from Humicola insolens and Chaetomium thermophilum, were secreted from S. cerevisiae at much higher levels than the other three, from Hypocrea jecorina, Phanerochaete chrysosporium and Talaromyces emersonii (FIG. 1). Because bands in the SDS-PAGE gel for the three weakly expressed candidate parents were difficult to discern, activity assays in which concentrated culture supernatants were incubated with phosphoric acid swollen cellulose (PASC) were performed to confirm the presence of active cellulase. The values for the reducing sugar formed, presented in FIG. 1, confirmed the presence of active CBH II in concentrated S. cerevisiae culture supernatants for all enzymes except T. emersonii CBH II. H. insolens and C. thermophilum sequences were chose to recombine with the most industrially relevant fungal CBH II enzyme, from H. jecorina. The respective sequence identities of the catalytic domains are 64% (1:2), 66% (2:3) and 82% (1:3), where H. insolens is parent 1, H. jecorina is parent 2 and C. thermophilum is parent 3. These respective catalytic domains contain 360, 358 and 359 amino acid residues.

Heterologous protein expression in the filamentous fungus H. jecorina, the organism most frequently used to produce cellulases for industrial applications, is much more arduous than in Saccharomyces cerevisiae. The observed secretion of H. jecorina CBH II from S. cerevisiae motivated the choice of this heterologous host. To minimize hyperglycosylation, which has been reported to reduce the activity of recombinant cellulases, the recombinant CBH II genes were expressed in a glycosylation-deficient dKRE2 S. cerevisiae strain. This strain is expected to attach smaller mannose oligomers to both N-linked and O-linked glycosylation sites than wild type strains, which more closely resembles the glycosylation of natively produced H. jecorina CBH II enzyme. SDS-PAGE gel analysis of the CBH II proteins, both with and without EndoH treatment to remove high-mannose structures, showed that EndoH treatment did not increase the electrophoretic mobility of the enzymes secreted from this strain, confirming the absence of the branched mannose moieties that wild type S. cerevisiae strains attach to glycosylation sites in the recombinant proteins.

The high resolution structure of H. insolens (pdb entry locn) was used as a template for SCHEMA to identify contacts that could be broken upon recombination. RASPP returned four candidate libraries, each with <E>below 15. The candidate libraries all have lower <E>than previously constructed chimera libraries, suggesting that an acceptable fraction of folded, active chimeras could be obtained for a relatively high <m>. Chimera sequence diversity was maximized by selecting the block boundaries leading to the greatest <m>=50. The blocks for this design are illustrated in FIG. 2B and detailed in Table 2.

TABLE 2
ClustalW multiple sequence alignment for parent CBH II enzyme catalytic domains.
Blocks 2, 4, 6 and 8 are denoted by boxes and grey shading. Blocks 1, 3, 5 and 7 are not
shaded. (H. inso: SEQ ID NO: 2; H. Jeco: SEQ ID NO: 4 and C. Ther: SEQ ID NO: 6).

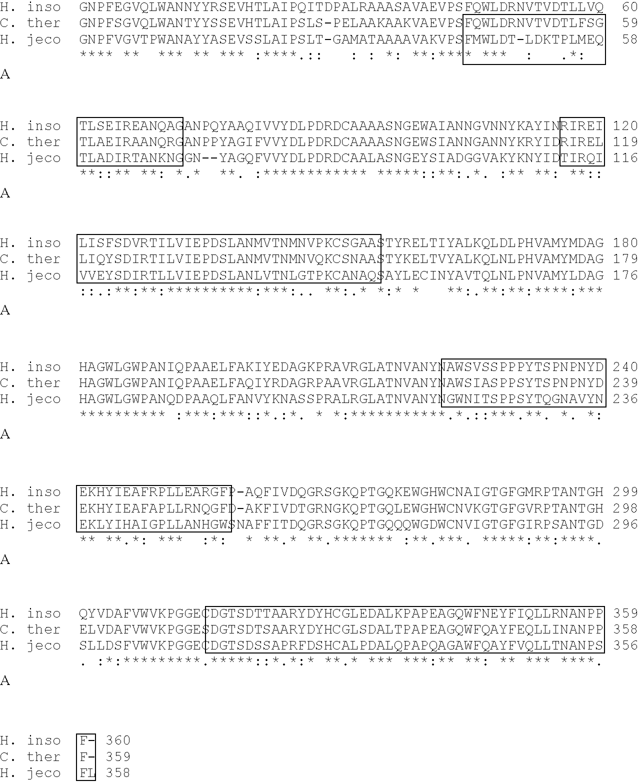

The H. insolens CBH II catalytic domain has an α/β barrel structure in which the eight helices define the barrel perimeter and seven parallel β-sheets form the active site (FIG. 2A). Two extended loops form a roof over the active site, creating a tunnel through which the substrate cellulose chains pass during hydrolysis. Five of the seven block boundaries fall between elements of secondary structure, while block 4 begins and ends in the middle of consecutive α-helices (FIGS. 2A, 2B). The majority of interblock sidechain contacts occur between blocks that are adjacent in the primary structure (FIG. 2C).

A sample set of 48 chimera genes was designed as three sets of 16 chimeras having five blocks from one parent and three blocks from either one or both of the remaining two parents (Table 3); the sequences were selected to equalize the representation of each parent at each block position. The corresponding genes were synthesized and expressed.

TABLE 3
Sequences of sample set CBH II enzyme chimeras.

| Inactive | Active |
|---|---|
| 13121211 | 11332333 |
| 12122221 | 21131311 |
| 33332321 | 31212111 |
| 33321331 | 22232132 |
| 21322232 | 33213332 |
| 21112113 | 23233133 |
| 31121121 | 13231111 |
| 32312222 | 12213111 |
| 23223223 | 31311112 |
| 31313323 | 11113132 |
| 32121222 | 13111313 |
| 12121113 | 21311131 |
| 22133222 | 11313121 |
| 33222333 | 21223122 |
| 11131231 | 22212231 |
| 11112321 | 23231222 |
| 12111212 | 32333113 |
| 31222212 | 12133333 |
| 22322312 | 13333232 |
| 12222213 | 33123313 |
| 12221122 | 21333331 |
| 22212323 | 23311333 |
| 23222321 | 33133132 |
| 32333223 | |
| 33331213 | |

Twenty-three of the 48 sample set S. cerevisiae concentrated culture supernatants exhibited hydrolytic activity toward PASC. These results suggest that thousands of the 6,561 possible CBH II chimera sequences (see e.g., Table 1) encode active enzymes. The 23 active CBH II sample set chimeras show considerable sequence diversity, differing from the closest parental sequence and each other by at least 23 and 36 amino acid substitutions and as many as 54 and 123, respectively. Their average mutation level <m>is 36.

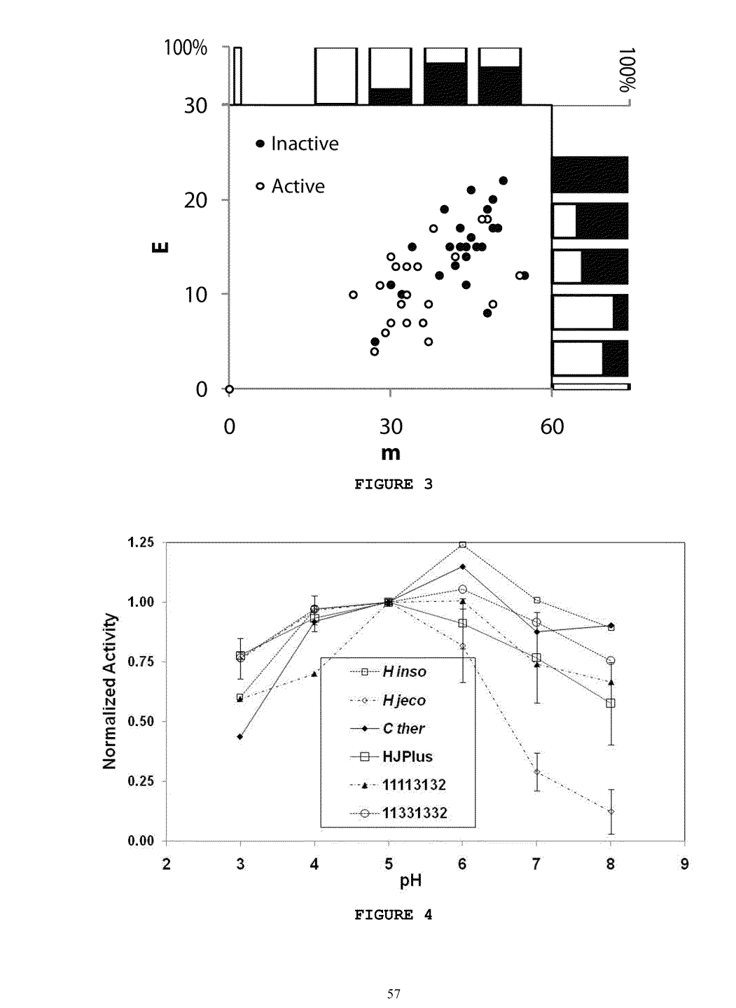

The correlations between E, m and the probability that a chimera is folded and active was analyzed. The amount of CBH II enzyme activity in concentrated expression culture supernatants, as measured by assaying for activity on PASC, was correlated to the intensity of CBH II bands in SDS-PAGE gels (FIG. 1). As with the H. jecorina CBH II parent, activity could be detected for some CBH II chimeras with undetectable gel bands. There were no observations of CBH II chimeras presenting gel bands but lacking activity. The probability of a CBH II chimera being secreted in active form was inversely related to both E and m (FIG. 3).

Half-lives of thermal inactivation (t_{1/2}) were measured at 63° C. for concentrated culture supernatants of the parent and active chimeric CBH II enzymes. The H. insolens, H. jecorina and C. thermophilum CBH II parent half-lives were 95, 2 and 25 minutes, respectively. The active sample set chimeras exhibited a broad range of half-lives, from less than 1 minute to greater than 3,000. Five of the 23 active chimeras had half-lives greater than that of the most thermostable parent, H. insolens CBH II.

In attempting to construct a predictive quantitative model for CBH II chimera half-life, five different linear regression data modeling algorithms were used (Table 4). Each algorithm was used to construct a model relating the block compositions of each sample set CBH II chimera and the parents to the log(t_{1/2}). These models produced thermostability weight values that quantified a block's contribution to log(t_{1/2}). For all five modeling algorithms, this process was repeated 1,000 times, with two randomly selected sequences omitted from each calculation, so that each algorithm produced 1,000 weight values for each of the 24 blocks. The mean and standard deviation (SD) were calculated for each block's thermostability weight. The predictive accuracy of each model algorithm was assessed by measuring how well each model predicted the tᵢₙs of the two omitted sequences. The correlation between measured and predicted values for the 1,000 algorithm iterations is the model algorithm's cross-validation score. For all five models, the cross-validation scores (X-val) were less than or equal to 0.57 (Table 4), indicating that linear regression modeling could not be applied to this small, 23 chimera t₁₁₂ data set for quantitative CBH II chimera half-life prediction.

TABLE 4
Cross validation values for application of 5 linear
regression algorithms to CBH II enzyme chimera block stability
scores.

| Method | Ridge | PLS | SVMR | LSVM | LPBoost |
|---|---|---|---|---|---|
| X-val | 0.56 | 0.55 | 0.50 | 0.42 | 0.43 |

Algorithm abbreviations:
ridge regression (RR),
partial least square regression (PLSR),
support vector machine regression (SVMR),
linear programming support vector machine regression (LPSVMR) and
linear programming boosting regression (LPBoostR).

Linear regression modeling was used to qualitatively classify blocks as stabilizing, destabilizing or neutral. Each block's impact on chimera thermostability was characterized using a scoring system that accounts for the thermostability contribution determined by each of the regression algorithms. For each algorithm, blocks with a thermostability weight value more than 1 SD above neutral were scored “+1”, blocks within 1 SD of neutral were assigned zero and blocks 1 or more SD below neutral were scored “−1”. A “stability score” for each block was obtained by summing the 1, 0, -1 stability scores from each of the five models. Table 5 summarizes the scores for each block. Block 1/parent 1 (B1P1), B6P3, B7P3 and B8P2 were identified as having the greatest stabilizing effects, while B1P3, B2P1, B3P2, B6P2, B7P1, B7P2 and B8P3 were found to be the most strongly destabilizing blocks.

TABLE 5
Qualitative block classification results generated by five linear
regression algorithms¹ for sample set CBH II enzyme chimeras.

| Block | Ridge | PLS | SVMR | LSVM | LPBoost | Sum |
|---|---|---|---|---|---|---|
| B1P1 | 1 | 0 | 1 | 1 | 0 | 3 |
| B1P2 | 0 | 0 | 0 | −1 | 0 | −1 |
| B1P3 | −1 | 0 | −1 | −1 | −1 | −4 |
| B2P1 | −1 | 0 | 0 | −1 | −1 | −3 |
| B2P2 | 1 | 0 | 0 | 0 | 0 | 1 |
| B2P3 | 1 | 0 | 0 | 0 | 0 | 1 |
| B3P1 | 1 | 0 | 1 | 0 | 0 | 2 |
| B3P2 | −1 | 0 | −1 | −1 | −1 | −4 |
| B3P3 | 1 | 0 | 1 | 0 | 0 | 2 |
| B4P1 | 0 | 0 | 0 | 0 | 0 | 0 |
| B4P2 | 0 | 0 | 0 | 0 | 0 | 0 |
| B4P3 | 0 | 0 | 0 | −1 | 0 | −1 |
| B5P1 | 0 | 0 | 0 | 0 | 0 | 0 |
| B5P2 | 0 | 0 | 0 | 0 | −1 | −1 |
| B5P3 | −1 | 0 | 0 | −1 | 0 | −2 |
| B6P1 | 1 | 0 | 0 | −1 | −1 | −1 |
| B6P2 | −1 | 0 | −1 | −1 | −1 | −4 |
| B6P3 | 1 | 1 | 1 | 1 | 1 | 5 |
| B7P1 | −1 | 0 | −1 | −1 | −1 | −4 |
| B7P2 | −1 | 0 | −1 | −1 | −1 | −4 |
| B7P3 | 1 | 0 | 1 | 1 | 1 | 4 |
| B8P1 | 1 | 0 | 1 | −1 | 0 | −1 |
| B8P2 | 1 | 0 | 1 | 1 | 0 | 3 |
| B8P3 | −1 | 0 | −1 | −1 | −1 | −4 |

Score of +1 denotes a block with thermostability weight (dimensionless metric for contribution of a block to chimera thermostability) greater than one standard deviation above neutral (stabilizing), score of 0 denotes block with weight within one standard deviation of neutral and −1 denotes block with weight more than one standard deviation below neutral (destabilizing).

In one embodiment of the disclosure, a chimera is provided that has a sum score from the contributions of each block/domain of greater than 0 using a qualitative block classification, wherein the qualitatively classify blocks are defined as stabilizing, destabilizing or neutral, wherein each block's impact on chimera thermostability is characterized using a scoring system that accounts for the thermostability contribution determined by a plurality of regression algorithms. For each algorithm, blocks with a thermostability weight value more than 1 SD above neutral were scored “+1”, blocks within 1 SD of neutral were assigned zero and blocks 1 or more SD below neutral were scored “−1”. A “stability score” for each block was obtained by summing the 1, 0, -1 stability scores from each of the five models.

A second set of genes encoding CBH II enzyme chimeras was synthesized in order to validate the predicted stabilizing blocks and identify cellulases more thermostable than the most stable parent. The 24 chimeras included in this validation set (Table 6) were devoid of the seven blocks predicted to be most destabilizing and enriched in the four most stabilizing blocks, where representation was biased toward higher stability scores. Additionally, the “HJP1us” 12222332 chimera was constructed by substituting the predicted most stabilizing blocks into the H. jecorina CBH II enzyme (parent 2).

TABLE 6
Sequences of 24 validation set CBH II enzyme chimeras,
nine of which were expressed in active form.

| Inactive | Active |
|---|---|
| 12122132 | 12111131 |
| 12132332 | 12132331 |
| 12122331 | 12131331 |
| 12112132 | 12332331 |
| 13122332 | 13332331 |
| 13111132 | 13331332 |
| 13111332 | 13311331 |
| 13322332 | 13311332 |
| 22122132 | 22311331 |
| 22322132 | |
| 22311332 | |
| 23111332 | |
| 23321131 | |
| 23321332 | |
| 23321331 | |

Concentrated supernatants of S. cerevisiae expression cultures for nine of the 24 validation set chimeras, as well as the HJP1us chimera, showed activity toward PASC (Table 6). Of the 15 chimeras for which activity was not detected, nine contained block B4P2. Of the 16 chimeras containing B4P2 in the initial sample set, only one showed activity toward PASC. Summed over both chimera sets and HJP1us, just two of 26 chimeras featuring B4P2 were active, indicating that this particular block is highly detrimental to expression of active cellulase in S. cerevisiae.

The stabilities of the 10 functional chimeric CBH II enzymes from the validation set were evaluated. Because the stable enzymes already had half-lives of more than 50 hours, residual hydrolytic activity toward PASC after a 12-hour thermal inactivation at 63° C. was used as the metric for preliminary evaluation. This 12-hour incubation produced a measurable decrease in the activity of the sample set's most thermostable chimera, 11113132, and completely inactivated the thermostable H. insolens parent CBH II. All ten of the functional validation set chimeras retained a greater fraction of their activities than the most stable parent, H. insolens CBH II.

TABLE 7
Specific activity values (ug glucose reducing sugar
equivalent/ug CBH II * hr) for three thermostable CBH II chimeras
and parents. Error is give as standard erros for between five
and eight replicates per CBH II. 2-hour reaction, 3 mg enzyme/g
PASC, 50° C., 25 mM sodium acetate, pH 4.8.

| | Ug Reducing |
|---|---|
| CBH II | Sugar/ug Enzyme * hr |
| Humicola insolens (Parent 1) | 2.4 ± 0.3 |
| Trichoderma reesei (Parent 2) | 7.5 ± 1.0 |
| Chaetomium thermophilium (Parent 3) | 3.0 ± 0.3 |
| TRPlus (Chimera 12222332) | 6.0 ± 0.5 |
| Chimera (11113132) | 2.7 ± 0.3 |
| Chimera (13311332) | 4.0 ± 0.2 |

TABLE 8
Half-lives of thermal inactivation for active CBH II sample
set chimeras at 63° C. Results for two independent
trials are presented.

| Chimera | t_{1/2} (min) | t_{1/2} (min) |
|---|---|---|
| H. insolens (P1) | 90 | 100 |
| T. reesei (P2) | 2 | 2 |
| C. thermophilum (P3) | 30 | 20 |
| 11113132 | 2800 | 3600 |
| 21333331 | 500 | 630 |
| 21311131 | 460 | 500 |
| 22232132 | 280 | 330 |
| 33133132 | 200 | 200 |
| 33213332 | 150 | 130 |
| 13333232 | 100 | 130 |
| 12133333 | 70 | 110 |
| 13231111 | 60 | 40 |
| 11313121 | 50 | 45 |
| 11332333 | 40 | 40 |
| 12213111 | 40 | 40 |
| 23311333 | 35 | 30 |
| 13111313 | 20 | 20 |
| 31311112 | 15 | 15 |
| 23231222 | 10 | 10 |
| 33123313 | 10 | 10 |
| 22212231 | 5 | 15 |
| 21223122 | 5 | 10 |
| 21131311 | 3 | 3 |
| 23233133 | 3 | 2 |
| 31212111 | 2 | 3 |
| 32333113 | <1 | <1 |

The activities of selected thermostable chimeras using purified enzymes was analyzed. The parent CBH II enzymes and three thermostable chimeras, the most thermostable sample set chimera 11113132, the most thermostable validation set chimera 13311332 and the HJPlus chimera 12222332, were expressed with C-terminal Hisₛ purification tags and purified. To minimize thermal inactivation of CBH II enzymes during the activity test, we used a shorter, two-hour incubation with the PASC substrate at 50° C., pH 4.8. As shown in Table 3, the parent and chimera CBH II specific activities were within a factor of four of the most active parent CBH II enzyme, from H. jecorina. The specific activity of HJPlus was greater than all other CBH II enzymes tested, except for H. jecorina CBH II.

The pH dependence of cellulase activity is also important, as a broad pH/activity profile would allow the use of a CBH II chimera under a wider range of potential cellulose hydrolysis conditions. H. jecorina CBH II has been observed to have optimal activity in the pH range 4 to 6, with activity markedly reduced outside these values. FIG. 4 shows that the H. insolens and C. thermophilum CBH II enzymes and all three purified thermostable CBH II chimeras have pH/activity profiles that are considerably broader than that of H. jecorina CBH II. Although Liu et al. report an optimal pH of 4 for C. thermophilum CBH II, the optimal pH of the recombinant enzyme here was near 7. Native H. insolens CBH II has a broad pH/activity profile, with maximum activity around pH 9 and approximately 60% of this maximal activity at pH 4. A similarly broad profile was observed for the recombinant enzyme. The HJPlus chimera has a much broader pH/activity profile than H. jecorina CBH II, showing a pH dependence similar to the other two parent CBH II enzymes.

Achieving activity at elevated temperature and retention of activity over extended time intervals are two primary motivations for engineering highly stable CBH II enzymes. The performance of thermostable CBH II chimeras in cellulose hydrolysis was tested across a range of temperatures over a 40-hour time interval. As shown in FIG. 5, all three thermostable chimeras were active on PASC at higher temperatures than the parent CBH II enzymes. The chimeras retained activity at 70° C., whereas the H. jecorina CBH II did not hydrolyze PASC above 57° C. and the stable H. insolens enzyme showed no hydrolysis above 63° C. The activity of HJP1us in long-time cellulose hydrolysis assays exceeded that of all the parents at their respective optimal temperatures.

While various specific embodiments have been illustrated and described, it will be appreciated that various changes can be made without departing from the spirit and scope of the invention(s).

What is claimed is:

1. A chimeric polypeptide comprising at least two domains from two different parental cellobiohydrolase II (CBH II) polypeptides, wherein the domains comprise from N- to C-terminus: (segment 1)-(segment 2)-(segment 3)-(segment 4)-(segment 5)-(segment 6)-(segment 7)-(segment 8);
wherein:
segment 1 comprises a sequence that is at least 50-100% identical to amino acid residue from about 1 to about x₁ of SEQ ID NO:2 (“1”), SEQ ID NO:4 (“2”) or SEQ ID NO:6 (“3”); segment 2 comprises a sequence that is at least 50-100% identical to amino acid residue x₁ to about x₂ of SEQ ID NO:2 (“1”), SEQ ID NO:4 (“2”) or SEQ ID NO:6 (“3”); segment 3 comprises a sequence that is at least 50-100% identical to amino acid residue x₂ to about x₃ of SEQ ID NO:2 (“1”), SEQ ID NO:4 (“2”) or SEQ ID NO:6 (“3”); segment 4 comprises a sequence that is at least 50-100% identical to amino acid residue x₃ to about x₄ of SEQ ID NO:2 (“1”), SEQ ID NO:4 (“2”) or SEQ ID NO:6 (“3”); segment 5 comprises a sequence that is at least 50-100% identical to about amino acid residue x₄ to about x₅ of SEQ ID NO:2 (“1”), SEQ ID NO:4 (“2”) or SEQ ID NO:6 (“3”); segment 6 comprises a sequence that is at least 50-100% identical to amino acid residue x₅ to about x₆ of SEQ ID NO:2 (“1”), SEQ ID NO:4 (“2”) or SEQ ID NO:6 (“3”); segment 7 comprises a sequence that is at least 50-100% identical to amino acid residue x₆ to about x₇ of SEQ ID NO:2 (“1”), SEQ ID NO:4 (“2”) or SEQ ID NO:6 (“3”); and segment 8 comprises a sequence that is at least 50-100% identical to amino acid residue x₇ to about x₈ of SEQ ID NO:2 (“1”), SEQ ID NO:4 (“2”) or SEQ ID NO:6 (“3”);
wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6
wherein the chimeric polypeptide has cellobiohydrolase activity and improved thermostability and/or pH stability compared to a CBH II polypeptide comprising SEQ ID NO:2, 4, or 6.

2. The polypeptide of claim 1, wherein segment 1 comprises amino acid residue from about 1 to about x₁ of SEQ ID NO:2 (“1”), SEQ ID NO:4 (“2”) or SEQ ID NO:6 (“3”) and having 1-10 conservative amino acid substitutions; segment 2 is from about amino acid residue x₁ to about x₂ of SEQ ID NO:2 (“1”), SEQ ID NO:4 (“2”) or SEQ ID NO:6 (“3”) and having about 1-10 conservative amino acid substitutions; segment 3 is from about amino acid residue x₂ to about x₃ of SEQ ID NO:2 (“1”), SEQ ID NO:4 (“2”) or SEQ ID NO:6 (“3”) and having about 1-10 conservative amino acid substitutions; segment 4 is from about amino acid residue x₃ to about x₄ of SEQ ID NO:2 (“1”), SEQ ID NO:4 (“2”) or SEQ ID NO:6 (“3”) and having about 1-10 conservative amino acid substitutions; segment 5 is from about amino acid residue x₄ to about x₅ of SEQ ID NO:2 (“1”), SEQ ID NO:4 (“2”) or SEQ ID NO:6 (“3”) and having about 1-10 conservative amino acid substitutions; segment 6 is from about amino acid residue x₅ to about x₆ of SEQ ID NO:2 (“1”), SEQ ID NO:4 (“2”) or SEQ ID NO:6 (“3”) and having about 1-10 conservative amino acid substitutions; segment 7 is from about amino acid residue x₆ to about x₇ of SEQ ID NO:2 (“1”), SEQ ID NO:4 (“2”) or SEQ ID NO:6 (“3”) and having about 1-10 conservative amino acid substitutions; and segment 8 is from about amino acid residue x₇ to about x₈ of SEQ ID NO:2 (“1”), SEQ ID NO:4 (“2”) or SEQ ID NO:6 (“3”) and having about 1-10 conservative amino acid substitutions.

3. The chimeric polypeptide of claim 1, wherein the chimeric polypeptide has at least one segment selected from the following:
segment 1 from SEQ ID NO:2; segment 6 from SEQ ID NO:6, segment 7 from SEQ ID NO:6 and segment 8 from SEQ ID NO:4.

4. The chimeric polypeptide of claim 3, wherein the chimeric polypeptide can be described as having segments 1X₂X₃X₄X₅332, wherein X₂ comprises a sequence that is at least 50-100% identical to amino acid residue x₁ to about x₂ of SEQ ID NO:2 (“1”), SEQ ID NO:4 (“2”) or SEQ ID NO:6 (“3”); X₃ comprises a sequence that is at least 50-100% identical to amino acid residue x₂ to about x₃ of SEQ ID NO:2 (“1”), SEQ ID NO:4 (“2”) or SEQ ID NO:6 (“3”); X₄ comprises a sequence that is at least 50-100% identical to amino acid residue x₃ to about x₄ of SEQ ID NO:2 (“1”), SEQ ID NO:4 (“2”) or SEQ ID NO:6 (“3”); X₅ comprises a sequence that is at least 50-100% identical to about amino acid residue x₄ to about x₅ of SEQ ID NO:2 (“1”), SEQ ID NO:4 (“2”) or SEQ ID NO:6 (“3”).

5. The chimeric polypeptide of claim 1, wherein the chimeric polypeptide comprises a segment structure selected from the group consisting of 11113132, 21333331, 21311131, 22232132, 33133132, 33213332, 13333232, 12133333, 13231111, 11313121, 11332333, 12213111, 23311333, 13111313, 31311112, 23231222, 33123313, 22212231, 21223122, 21131311, 23233133, 31212111, 12222332 and 32333113.

6. The chimeric polypeptide of claim 1, wherein the cimeric polypeptide comprises a segment structure selected from the group set forth in Table 1.

7. A polynucleotide encoding a polypeptide of claim 1.

8. A vector comprising a polynucleotide of claim 7.

9. A host cell comprising the vector of claim 8 or the polynucleotide of claim 7.

10. An enzymatic preparation comprising a polypeptide of claim 1.

11. An enzymatic preparation comprising a polypeptide produced by a host cell of claim 9.

12. A method of treating a biomass comprising cellulose, the method comprising contacting the biomass with a polypeptide of claim 1.

13. A method of treating a biomass comprising cellulose, the method comprising contacting the biomass with a host cell of claim 9.

14. A method of generating a thermostable chimeric cellobiohydrolase polypeptide, comprising recombining segments from at least 3 parental cellobiohydrolase polypeptide wherein the chimeric polypeptide comprises from N- to C-terminus 8 segments wherein:
segment 1 comprises a sequence that is at least 50-100% identical to amino acid residue from about 1 to about x₁ of SEQ ID NO:2 (“1”), SEQ ID NO:4 (“2”) or SEQ ID NO:6 (“3”); segment 2 comprises a sequence that is at least 50-100% identical to amino acid residue x₁ to about x₂ of SEQ ID NO:2 (“1”), SEQ ID NO:4 (“2”) or SEQ ID NO:6 (“3”); segment 3 comprises a sequence that is at least 50-100% identical to amino acid residue x₂ to about x₃ of SEQ ID NO:2 (“1”), SEQ ID NO:4 (“2”) or SEQ ID NO:6 (“3”); segment 4 comprises a sequence that is at least 50-100% identical to amino acid residue x₃ to about x₄ of SEQ ID NO:2 (“1”), SEQ ID NO:4 (“2”) or SEQ ID NO:6 (“3”); segment 5 comprises a sequence that is at least 50-100% identical to about amino acid residue x₄ to about x₅ of SEQ ID NO:2 (“1”), SEQ ID NO:4 (“2”) or SEQ ID NO:6 (“3”); segment 6 comprises a sequence that is at least 50-100% identical to amino acid residue x₅ to about x₆ of SEQ ID NO:2 (“1”), SEQ ID NO:4 (“2”) or SEQ ID NO:6 (“3”); segment 7 comprises a sequence that is at least 50-100% identical to amino acid residue x₆ to about x₇ of SEQ ID NO:2 (“1”), SEQ ID NO:4 (“2”) or SEQ ID NO:6 (“3”); and segment 8 comprises a sequence that is at least 50-100% identical to amino acid residue x₇ to about x₈ of SEQ ID NO:2 (“1”), SEQ ID NO:4 (“2”) or SEQ ID NO:6 (“3”);
wherein x₁ is residue 43, 44, 45, 46, or 47 of SEQ ID NO:2, or residue 42, 43, 44, 45, or 46 of SEQ ID NO:4 or SEQ ID NO:6; x₂ is residue 70, 71, 72, 73, or 74 of SEQ ID NO:2, or residue 68, 69, 70, 71, 72, 73, or 74 of SEQ ID NO:4 or SEQ ID NO:6; x₃ is residue 113, 114, 115, 116, 117 or 118 of SEQ ID NO:2, or residue 110, 111, 112, 113, 114, 115, or 116 of SEQ ID NO:4 or SEQ ID NO:6; x₄ is residue 153, 154, 155, 156, or 157 of SEQ ID NO:2, or residue 149, 150, 151, 152, 153, 154, 155 or 156 of SEQ ID NO:4 or SEQ ID NO:6; x₅ is residue 220, 221, 222, 223, or 224 of SEQ ID NO:2, or residue 216, 217, 218, 219, 220, 221, 222 or 223 of SEQ ID NO:4 or SEQ ID NO:6; x₆ is residue 256, 257, 258, 259, 260 or 261 of SEQ ID NO:2, or residue 253, 254, 255, 256, 257, 258, 259 or 260 of SEQ ID NO:4 or SEQ ID NO:6; x₇ is residue 312, 313, 314, 315 or 316 of SEQ ID NO:2, or residue 309, 310, 311, 312, 313, 314, 315 or 318 of SEQ ID NO:4 or SEQ ID NO:6; and x₈ is an amino acid residue corresponding to the C-terminus of the polypeptide have the sequence of SEQ ID NO:2, SEQ ID NO:4 or SEQ ID NO:6;
screening the chimeric polypeptide for the ability to hydrolyze cellulose at a temperature of about 63° C.

15. A polypeptide identified by the method of claim 14.


SEQUENCE LISTING (11 sequences)

SEQ ID NO: 1 - DNA, 1083 bp, Humicola insolens
  CDS (1)..(1083)
ggtaacccctttgaaggtgttcagctgtgggctaataactattatagatctgaggtacatacactggccattccgcaaattacagaccccgcgttgcgtgccgcagctagtgctgtggctgaggtgccaagttttcaatggctggacagaaatgtaacagtggatactttgttggtacagactttgtcagaaatccgtgaggccaatcaagcaggtgctaatccccaatatgcagcgcaaatcgtggtctatgatctgcccgatagagactgtgcagctgccgcctcgaatggtgaatgggcaatagcgaacaacggtgtaaacaattacaaagcttacattaatagaattagagagatattgataagtttttcggacgttagaacgatattagtcattgagccagatagtctagctaatatggtcacaaatatgaatgtcccgaagtgttccggtgcagccagcacttatagggaattaaccatatatgcactgaagcaattggatctgcctcatgtcgctatgtacatggatgccggccacgctggatggttaggctggccggcaaacattcagccagccgcagaattgtttgccaaaatttacgaagatgctggaaagcctagagcagtgagaggtcttgcaactaatgttgctaattacaatgcatggtcagtttcatcccctccaccatacacaagtccaaatccaaactacgatgaaaagcattatatcgaagcattcagacccttattagaagcccgtggtttcccagcccaatttatagtggatcagggaagatcaggtaagcaaccaactggccaaaaggagtgggggcattggtgtaatgctattggcacaggatttggtatgagacctactgctaataccggtcaccagtatgtggatgcttttgtttgggttaaaccgggcggtgaatgcgacgggaccagcgatactacggcggccagatatgattatcattgtggtctggaagatgcattaaaaccagctcctgaagccggccagtggttcaacgaatacttcattcaattgcttaggaacgctaacccgcccttttaa

SEQ ID NO: 2 - protein, 360 aa, Humicola insolens
GNPFEGVQLWANNYYRSEVHTLAIPQITDPALRAAASAVAEVPSFQWLDRNVTVDTLLVQTLSEIREANQAGANPQYAAQIVVYDLPDRDCAAAASNGEWAIANNGVNNYKAYINRIREILISFSDVRTILVIEPDSLANMVTNMNVPKCSGAASTYRELTIYALKQLDLPHVAMYMDAGHAGWLGWPANIQPAAELFAKIYEDAGKPRAVRGLATNVANYNAWSVSSPPPYTSPNPNYDEKHYIEAFRPLLEARGFPAQFIVDQGRSGKQPTGQKEWGHWCNAIGTGFGMRPTANTGHQYVDAFVWVKPGGECDGTSDTTAARYDYHCGLEDALKPAPEAGQWFNEYFIQLLRNANPPF

SEQ ID NO: 3 - DNA, 1077 bp, Hypocrea jecorina
  CDS (1)..(1077)
ggtaatccattcgttggggtgacaccctgggcgaacgcctattatgcttctgaggtttcatccctagctattccctctttaacaggtgcaatggctacagccgccgctgccgttgcaaaggtcccttccttcatgtggctggatactttggacaaaacccccttaatggaacaaacgttggctgatatacgtactgcgaataaaaacggcggcaattatgctggacaatttgtggtttatgacctgccggatagagattgtgctgcactagcgagcaacggggagtacagcattgcggatggcggtgtcgcaaagtacaaaaactatatagatactatcaggcaaatagttgtcgaatacagtgatattcgtacgctgcttgtaatcgaacccgattccttagcgaacttggtaacaaatctaggtactccgaagtgtgcgaacgcgcagagtgcttatcttgagtgcatcaattatgcagtcacccagttgaatttgccaaacgttgcaatgtatcttgatgctggtcatgccgggtggttgggttggccagcaaatcaggatcccgctgcgcagctgtttgcaaatgtttacaaaaatgcctcaagtcctagagcgctgaggggtcttgcaacaaatgttgctaattacaacggatggaatattacctcacccccatcatacacacaaggaaatgctgtttacaatgaaaaactttatattcatgccattggtccactgctggctaatcacggatggagtaatgcctttttcattacagatcaagggagaagtggtaaacaacctactggacaacaacaatggggtgactggtgtaatgttatcggtactgggtttggcatcagaccatcagcgaatacgggtgattcattgttggactcatttgtttgggttaaacccgggggtgaatgtgatggaacgagtgattcttctgctccaaggttcgattctcattgcgcattaccagatgctttgcagccagcacctcaagcaggagcttggttccaagcttattttgtacaattactgactaacgccaatcctagttttctataa

SEQ ID NO: 4 - protein, 358 aa, Hypocrea jecorina
GNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 5 - DNA, 1077 bp, Chaetomium thermophilium
  CDS (1)..(1077)
ggtaaccctttcagtggtgtgcagttatgggctaatacttactattcttcagaagtccacaccttagctatcccaagcttaagtccagaattagcggctaaggcggcgaaagtagctgaagtgccatcattccaatggttagatagaaacgtgactgtggatactctgttttctggtacacttgctgagataagggcggctaaccaaaggggagctaatccaccatatgctggcatctttgtggtttatgaccttcctgatagagattgtgctgccgctgcaagcaatggtgaatggagtatagctaacaacggtgctaacaactataagagatatatcgatagaattagagaattgttgattcagtactcagatatcaggacaattttggttattgaaccagacagtctagcaaatatggttactaacatgaacgtacaaaaatgttctaacgcagcatctacgtataaagaactgactgtgtatgcattgaaacagttgaacttgccacacgtagccatgtatatggatgcaggtcacgccggctggttaggctggcccgctaatatacagcctgccgcagaattattcgcgcaaatatacagagacgctggacgtccggctgcggtcaggggtcttgccactaacgttgcaaattacaacgcttggtcaatagcgagtcctccatcgtacacaagccctaacccaaactacgatgagaagcattacatagaagcatttgctcctttgcttcgtaaccaaggttttgatgcaaagtttatcgtcgataccggaagaaacggcaagcagccgacagggcagctagaatgggggcactggtgcaatgtcaagggtacgggtttcggtgttagacccacggctaacactgggcatgagttggttgatgcattcgtttgggtaaaacccggaggagagtcagacggtacttctgatactagtgctgccagatacgattaccactgtggcctttctgatgctttgacaccagcccctgaagccgggcaatggttccaggcctacttcgaacaactattgattaacgcaaacccaccatag

SEQ ID NO: 6 - protein, 358 aa, Chaetomium thermophilium
GNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNYKRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSDTSAARYDYHCGLSDALTPAPEAGQWFQAYFEQLLINANPP

SEQ ID NO: 7 - DNA, 267 bp, Artificial Sequence
  CBD Linker
gctagctgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattca

SEQ ID NO: 8 - protein, 89 aa, Artificial Sequence
  Synthetic Construct
ASCSSVWGQCGGQNWSGPTCCASGSTCVYSNDYYSQCLPGAASSSSSTRAASTTSRVSPTTSRSSSATPPPGSTTTRVPPVGSGTATYS

SEQ ID NO: 9 - DNA, 21 bp, Artificial Sequence
  Oligonucleotide Primer CBH2L
gctgaacgtgtcatcggttac

SEQ ID NO: 10 - DNA, 23 bp, Artificial Sequence
  Oligonucleotide Primer RSQ3080
gcaacacctggcaattccttacc

SEQ ID NO: 11 - DNA, 24 bp, Artificial Sequence
  Oligonucleotide Primer CBH2LPCR
gctgaacgtgtcatcgttacttag